(19) United States
(12) Patent Application Publication
(10) Pub. No.: US 2025/0065316 A1
(43) Pub. Date: Feb. 27, 2025

(54) Rhenium(V) Alkylidyne Complexes and Methods of Use and Preparation Thereof

(71) Applicant: The Hong Kong University of Science and Technology, Hong Kong (CN)

(72) Inventors: Guochen JIA, Hong Kong (CN); Mingxu CUI, Hong Kong (CN); Jie HUANG, Hong Kong (CN)

(21) Appl. No.: 18/802,024

(22) Filed: Aug. 13, 2024

Related U.S. Application Data
(60) Provisional application No. 63/519,832, filed on Aug. 15, 2023.

(51) Int. Cl. B01J 31/24; B01J 31/22; C07C 29/36; C07C 45/68; C07D 211/06; C07D 225/02; C07D 321/00; C07D 487/22

(52) U.S. Cl. CPC B01J 31/2404; B01J 31/2265; C07C 29/36; C07C 45/68; C07D 211/06; C07D 225/02; C07D 321/00; C07D 487/22; B01J 2231/546; B01J 2531/74

(57) ABSTRACT

Re(V) alkylidyne-based catalytic systems for alkyne metathesis reactions capable of effecting alkyne metathesis, such as homo- and cross-metathesis of alkynes or diynes, ring closing metathesis and ring-opening metathesis, at room temperature to moderate temperatures. The catalysts are stable to air and moisture and tolerate a variety of functional groups in substrates and can be readily prepared on a large scale, and thus are practical catalytic systems of Re(V) alkylidynes.

### CROSS-REFERENCE TO RELATED APPLICATIONS

The present application claims priority from U.S. Provisional Patent Application No. 63/519,832, filed on Aug. 15, 2023, which is hereby incorporated by reference in its entirety.

### TECHNICAL FIELD

The present disclosure generally relates to Re(V) alkylidyne-based catalytic systems for alkyne metathesis and methods of use thereof.

### BACKGROUND

As a synthetically very useful transformation, transition metal catalyzed alkyne metathesis is finding growing practical applications in areas such as organic synthesis, polymerization, as well as dynamic covalent chemistry. There has been much interest in the development of catalytic systems that are highly efficient, easily accessible, functional group tolerant and user-friendly. Early studies mainly focused on heterogeneous mixtures of inorganic oxides, such as WO₃ and MoO₃ with silica or the homogeneous mixture of Mo(CO)₆ and phenol. These ill-defined in-situ catalytic systems can promote alkyne metathesis reactions, but are of low efficiency (requiring activation at high temperatures (>150° C.) and have very limited substrate functional group compatibility.

Recent works in the catalyst development have mainly been focused on high-valent d⁰ metal alkylidyne systems. These efforts have led to the invention of a library of outstanding catalysts based on well-defined or in situ generated W(VI) and Mo(VI) alkylidyne complexes. These catalytic systems can catalyze alkyne metathesis reactions in high efficiency. However, these high-valent d⁰ W(VI) and Mo(VI) alkylidyne complexes are usually very air and moisture sensitive and have limited substrate functional group compatibility.

Rhenium(V) alkylidyne complexes that can promote alkyne metathesis reactions have been described in U.S. Patent No. US20230059662A1. Although the pyridine or phosphine complexes disclosed in the above patent application exhibit reasonable metathesis activity and unprecedented stability towards air, moisture and functional groups, they have the following two drawbacks: 1) Compared with highly efficient d° Mo(VI) and W(VI) alkylidyne catalysts, the these Re(V) alkylidyne complexes are less active and the catalytic alkyne metathesis reactions require harsher conditions (e.g., 100° C.) to complete in a reasonably short period. 2) The preparation of pyridine-coordinate complexes can hardly be scaled up due to technical issues. This becomes one of the key obstacles for the commercialization of Re(V) alkyne metathesis catalysts.

To address these deficiency, an ideal alkyne metathesis catalyst based on Re(V) alkylidyne complexes should have the following features: 1) the catalyst should be stable to air and moisture to improve ease of use and storage; 2) it should be robust enough to tolerate a wide variety of functional groups; 3) it should be active enough to catalyze metathesis reactions at moderate temperatures or even at ambient temperature; and 4) it can be easily prepared on scale from commercially available chemicals.

### SUMMARY

The present disclosure meets the above requirements and provides two Re(V)-alkylidyne based catalytic systems for alkyne metathesis reactions, which are significantly more active than previously reported catalytic systems. The aqua complex Re(≡CCH₂Ph)(^{Ph}PO)₂(H₂O) can even effect alkyne metathesis at room temperature. The in situ catalytic system composed of Re(≡CCH₂Ph)(^{Ph}PO)₂(PMePh₂) and [(p-cymene)RuCl₂]₂ is able to promote alkyne metathesis at moderate temperatures. They can efficiently catalyze alkyne homometathesis, cross-metathesis (ACM), ring-closing alkyne metathesis (RCAM), acyclic diyne metathesis macrocyclization (ADIMAC) and ring-opening alkyne metathesis polymerization (ROAMP) with good functional group tolerance. Both systems are derived from the complex Re(≡CCH₂Ph)(^{Ph}PO)₂(PMePh₂) which is air stable and can be easily prepared in a large scale from the commercially available phosphine Ph₂P(o-C₆H₄—OH). Thus, they represent practical alkyne metathesis catalytic systems based on Re(V)-alkylidynes.

In a first aspect, provided herein is a rhenium(V) alkylidyne complex of Formula 1:

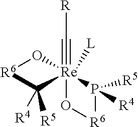

wherein L is H₂O, an ether, a ketone, a furan, a thioether, a thiophene, a tertiary amine, or a salt selected from the group consisting of a metal halide, a metal alkoxide, a metal thiolate and a metal amide;
R is alkyl, aryl, thiophene, or CH₂Ari, wherein Ar¹ is aryl;
each of R⁴ and R⁵ is independently alkyl, cycloalkyl or aryl; and
R⁶ for each instance is independently alkyl, alkenyl or aryl.

In certain embodiments, L is H₂O, tetrahydrofuran, tetrahydropyran, or Et₂O.

In certain embodiments, R is CH₂ArO, wherein Ar¹ is phenyl or o-bromophenyl.

In certain embodiments, each of R⁴ and R⁵ is independently alkyl, cycloalkyl, or phenyl optionally substituted with one or more substituents selected from alkyl, CH₃, OCH₃, F, and CF₃.

In certain embodiments, each of R⁴ and R⁵ is cyclohexyl, phenyl, 4-fluorophenyl, 2,4-difluorophenyl, 4-trifluoromethylphenyl, 2,4-bis(trifluoromethyl)phenyl, 2,4-dimethylphenyl, or 2,4-dimethoxyphenyl.

In certain embodiments, R⁶ is phenyl optionally substituted with alkyl or trifluoromethyl.

In certain embodiments, R⁶ is a moiety selected from the group consisting of:

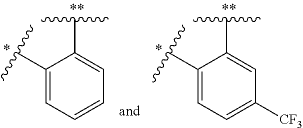 [SMILES: *c1ccccc1* ; *c1ccc(C(F)(F)F)cc1*]

wherein * represents the site of the covalent bond to oxygen and ** represents the site of the covalent bond to phosphorous.

In certain embodiments, the rhenium(V) alkylidyne complex is selected from the group consisting of:

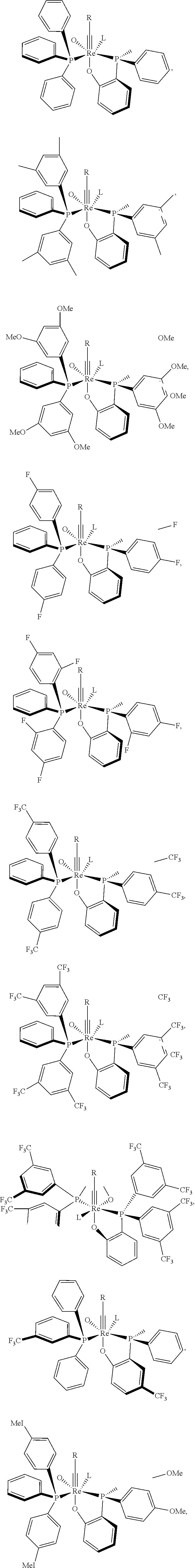

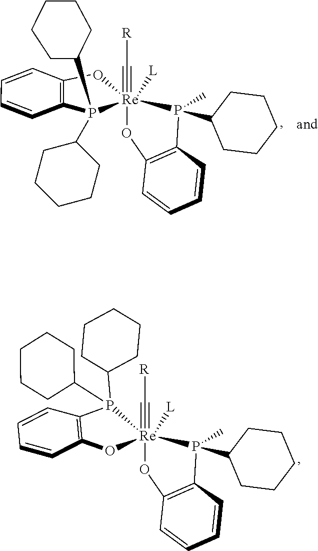

wherein R is alkyl, aryl, 2-thiophene, or CH₂Ar¹, wherein Ar¹ is phenyl or o-bromophenyl; and L is H₂O, tetrahydrofuran, tetrahydropyran, or Et₂O.

In certain embodiments, R is CH₂Ph.

In certain embodiments, L is H₂O.

In certain embodiments, the rhenium(V) alkylidyne complex has the structure.

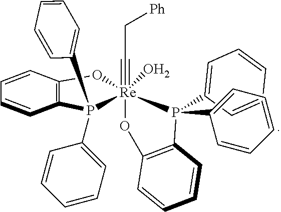

In a second aspect, provided herein is a method of preparing the rhenium (V) alkylidyne complex described herein, the method comprising: contacting a catalyst precursor of Formula 2:

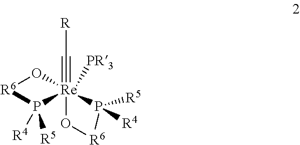

wherein R is alkyl, aryl, thiophene, or CH₂Ar¹, wherein Ar¹ is aryl;
PR′₃ is PMePh₂ or PMe₂Ph;
each of R⁴ and R⁵ is independently alkyl, cycloalkyl, or aryl; and
R⁶ for each instance is independently alkyl, alkenyl or aryl;
with a ligand selected from the group consisting of H₂O and an alkyl ether; and a phosphine scavenger selected from CuCl, CuI, or [(p-cymene]RuCl₂]₂; thereby forming the rhenium (V) alkylidyne complex.

In a third aspect, provided herein is a method for performing a metathesis reaction, the method comprising: contacting a rhenium(V) alkylidyne complex described herein with at least one alkyne substrate thereby forming an alkyne metathesis product.

In certain embodiments, the at least one alkyne substrate is selected from the group consisting of an acyclic alkyne, a cyclic alkyne, an acyclic diyne, and a cyclic diyne.

In a fourth aspect, provided herein is a method for performing a metathesis reaction, the method comprising: contacting a catalyst precursor of Formula 2, a phosphine scavenger, and at least one alkyne substrate thereby forming an alkyne metathesis product, wherein the catalyst precursor of Formula 2 has the structure:

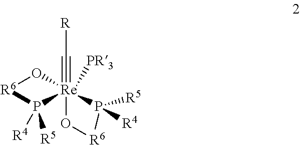

wherein R is alkyl, aryl, thiophene, or CH₂Ari, wherein Ar¹ is aryl;
PR′₃ is PMePh₂ or PMe₂Ph;
each of R⁴ and R⁵ is independently alkyl, cycloalkyl, or aryl; and
R⁶ for each instance is independently alkyl, alkenyl or aryl; and the phosphine scavenger is selected from [(p-cymene]RuCl₂]₂, [(p-cymene]OsCl₂]₂, [RhCl(COD)]₂, IrCl(COD)]₂, PdCl₂(COD), CuCl, or CuI.

In certain embodiments, R is CH₂ArO, wherein Ar¹ is phenyl or o-bromophenyl.

In certain embodiments, each of R⁴ and R⁵ is cyclohexyl, phenyl, 4-fluorophenyl, 2,4-difluorophenyl, 4-trifluoromethylphenyl, 2,4-bis(trifluoromethyl)phenyl, 2,4-dimethylphenyl, or 2,4-dimethoxyphenyl.

In certain embodiments, R⁶ is phenyl optionally substituted with alkyl or trifluoromethyl.

In certain embodiments, R⁶ is a moiety selected from the group consisting of:

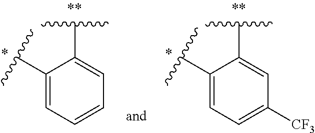 [SMILES: *c1ccccc1* ; *c1ccc(C(F)(F)F)cc1*]

wherein * represents the site of the covalent bond to oxygen and ** represents the site of the covalent bond to phosphorous.

In certain embodiments, the phosphine scavenger is [(p-cymene]RuCl₂]₂ and the catalyst precursor is:

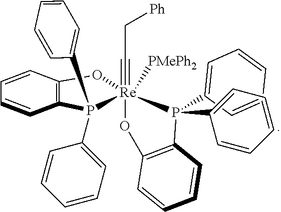

In a fifth aspect provided herein is a rhenium(V) alkylidyne complex having the structure:

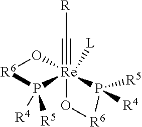

In certain embodiments, L is weakly coordinating ligand, such as H₂O, THF, or Et₂O.

In certain embodiments, R is CH₂ArO, wherein Ar¹ is phenyl or o-bromophenyl.

In certain embodiments, each of R⁴ and R⁵ is independently alkyl, cycloalkyl, or phenyl optionally substituted with one or more substituents selected from alkyl, CH₃, OCH₃, F, and CF₃.

In certain embodiments, each of R⁴ and R⁵ is cyclohexyl, phenyl, 4-fluorophenyl, 2,4-difluorophenyl, 4-trifluoromethylphenyl, 2,4-bis(trifluoromethyl)phenyl, 2,4-dimethylphenyl, or 2,4-dimethoxyphenyl.

In certain embodiments, R⁶ is alkyl, alkenyl or phenyl optionally substituted with alkyl or trifluoromethyl.

In certain embodiments, R⁶ is a moiety selected from the group consisting of

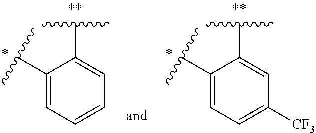 [SMILES: *c1ccccc1* ; *c1ccc(C(F)(F)F)cc1*]

wherein * represents the site of the covalent bond to oxygen and ** represent the site of the covalent bond to phosphorous.

In certain embodiments, the rhenium(V) alkylidyne complex has the structure:

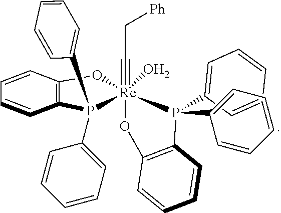

Compared with Re(V) alkylidyne catalysts which have been described in U.S. Patent No. US20230059662A1. The aqua complexes disclosed here have the following advantages: 1) Aqua complexes are easier to be synthesized and could be prepared in large scale (At least 1 gram scale). 2) The aqua complex is significantly more active in catalyzing alkyne metathesis reactions than pyridine complex. As shown in FIG. 1, the homometathesis of 1-methoxy-4-(1-propyn-1-yl)benzene (15), catalyzed by the aqua complex 14 at 100° C. in the presence of 5 A molecular sieves (MS) could go completion within 5 min. In contrast, the reaction with phosphine complex 11 and the pyridine complex 8b achieved conversions of less than 5% and ca. 50% respectively even after 2 h. 3) The aqua complex 14 can catalyze alkyne metathesis reactions at room temperature. As shown in FIG. 2, entry 3, the model reaction also proceeded to 92% conversion in 16 h at room temperature (25° C.). For comparison, the pyridine complex 8b, which has been described in U.S. Patent No. US20230059662A1 cannot catalyze the model reaction under the same conditions, shown in FIG. 2.

In a sixth aspect, provided herein is a method of preparing the rhenium (V) alkylidyne complex described herein, the method comprising: contacting a catalyst precursor of Formula 2:

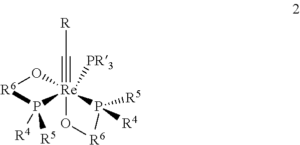

wherein R is alkyl, aryl, thiophene, or CH₂Ar¹, wherein Ar¹ is aryl;
PR′₃ is PMePh₂ or PMe₂Ph;
each of R⁴ and R⁵ is independently alkyl, cycloalkyl, or aryl; and
R⁶ for each instance is independently alkyl, alkenyl or aryl;
with a phosphine scavenger selected from late transition metal complexes, including but not limited to [(p-cymene]RuCl₂]₂, [(p-cymene]OsCl₂]₂, [RhCl(COD)]₂, IrCl(COD)]₂, PdCl₂(COD), and a weakly coordinating ligand compound such as H₂O, THE or Et₂O thereby forming the rhenium (V) alkylidyne complex.

In a seventh aspect, provided herein is a method of using a catalyst precursor, the method comprising: contacting a catalyst precursor of Formula 2 and a phosphine scavenger, wherein the catalyst precursor of Formula 2 is represented by the chemical structure:

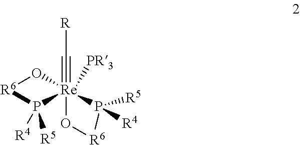

wherein R is alkyl, aryl, thiophene, or CH₂Ari, wherein Ar¹ is aryl;
PR′₃ is PMePh₂ or PMe₂Ph;
each of R⁴ and R⁵ is independently alkyl, cycloalkyl, or aryl; and
R⁶ for each instance is independently alkyl, alkenyl or aryl; and
the phosphine scavenger is selected from late transition metal complexes, such as [(p-cymene]RuCl₂]2, [(p-cymene]OsCl₂]2 [RhCl(COD)]₂, IrCl(COD)]₂, and PdCl₂(COD). In certain embodiments, the catalyst precursor and the phosphine scavenger are contacted in a molar ratio of 1:0.5, respectively. In certain embodiments, the catalyst precursor of Formula 2 and the phosphine scavenger are contacted in the presence of at least one alkyne substrate. In certain embodiments, the catalyst precursor of Formula 2 and the phosphine scavenger are contacted in the absence of any ligands, such as pyridine.

Compared with pyridine complexes which have been described in U.S. Patent No. US20230059662A1 the new method that using the 1:0.5 in situ mixture of Re(V) alkylidynes with the formula 6 and phosphine scavengers disclosed here have the following advantages: 1) This is a more convenient method of using Re(V) alkylidyne complexes for alkyne metathesis reactions. By using this method, there is no need to make pyridine complexes which are hard to make. 2) The in situ mixtures are significantly more active in catalyzing alkyne metathesis reactions than pyridine complexes or PMePh₂ complexes. As shown in FIG. 1, the homometathesis of 1-methoxy-4-(1-propyn-1-yl)benzene (15), catalyzed by the 1:0.5 mixture of complex 11/[(p-cymene)RuCl₂]₂ at 100° C. in the presence of 5 A molecular sieves (MS) could go completion within 30 min. In contrast, the reaction with phosphine complex 11 and the pyridine complex 8b achieved conversions of less than 5% and ca. 50% respectively even after 2 h. 3) The in situ mixture can catalyzed alkyne metathesis in mild conditions. As shown in FIG. 2, the model reaction catalyzed by the mixture of 11 and [(p-cymene)RuCl₂]₂ achieved full conversion within 12 h at 60° C.

In an eighth aspect, provided herein is a method for performing a metathesis reaction, the method comprising: contacting a rhenium(V) alkylidyne complex of the fifth aspect with at least one alkyne substrate thereby forming an alkyne metathesis product.

In certain embodiments, the metathesis reaction is an alkyne homo-metathesis reaction, alkyne cross-metathesis, ring closing alkyne metathesis, ring opening alkyne metathesis polymerization, or acyclic diyne metathesis macrocyclozation.

In certain embodiments, the at least one alkyne substrate is selected from the group consisting of an acyclic alkyne, a cyclic alkyne, an acyclic diyne, and a cyclic diyne.

### BRIEF DESCRIPTION OF THE DRAWINGS

The above and other objects and features of the present disclosure will become apparent from the following description of the disclosure, when taken in conjunction with the accompanying drawings.

FIG. 1 depicts a diagram showing catalyst testing data, wherein 11 is Re(≡CCH₂Ph)(^{Ph}PO)₂(PMePh₂), 8b is Re(≡CCH₂Ph)(^{Ph}PO)₂(py), 14 is Re(≡CCH₂Ph)(^{Ph}PO)₂(H₂O). Conditions: Substrate (1-methoxy-4-(1-propyn-1-yl)benzene, 0.3 mmol), catalyst(s) (5 mol %), 5 A MS (450 mg), dry toluene (3 mL), 100° C. The yields were determined by 1H NMR using CH₂Ph₂ as an internal standard.

FIG. 2 depicts a diagram showing catalyst testing data at mild conditions, wherein 11 is Re(≡CCH₂Ph)(^{Ph}PO)₂(PMePh₂), 14 is Re(≡CCH₂Ph)(^{Ph}PO)₂(H₂O), 8b is Re(≡CCH₂Ph)(^{Ph}PO)₂(py). Standard condition: 1-methoxy-4-(1-propyn1-yl)benzene (15, 0.3 mmol), catalyst(s) (5 mol %), 5 A MS (450 mg), dry toluene (3 mL). All reagents and catalysts were weighed in air. The yields were determined by ¹H NMR using CH₂Ph₂ as an internal standard. [a]0.5 mol % catalyst loading.

FIG. 3 depicts an Oak Ridge thermal ellipsoid plot (ORTEP) of complex Re(≡CCH₂Ph)(^{Ph}PO)₂(H₂O)

### DETAILED DESCRIPTION

### Definitions

The following terms shall be used to describe the present invention. In the absence of a specific definition set forth herein, the terms used to describe the present invention shall be given their common meaning as understood by those of ordinary skill in the art.

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

As used herein, unless otherwise indicated, the term “halo” or “halide” includes fluoro, chloro, bromo or iodo.

As used herein, “alkyl” refers to a straight-chain or branched saturated hydrocarbon group. Examples of alkyl groups include methyl-, ethyl-, propyl (e.g., n-propyl and isopropyl), butyl (e.g., n-butyl, iso-butyl, sec-butyl, tert-butyl), pentyl groups (e.g., 1-methylbutyl, 2-methylbutyl, iso-pentyl, tert-pentyl, 1,2-dimethylpropyl, neopentyl, and 1-ethylpropyl), hexyl groups, and the like. In various embodiments, an alkyl group can have 1 to 40 carbon atoms (i.e., C1-40 alkyl group), for example, 1-30 carbon atoms (i.e., C1-30 alkyl group). In certain embodiments, an alkyl group can have 1 to 6 carbon atoms, and can be referred to as a “lower alkyl group.” Examples of lower alkyl groups include methyl, ethyl, propyl (e.g., n-propyl and isopropyl), and butyl groups (e.g., n-butyl, isobutyl, sec-butyl, tert-butyl). In certain embodiments, alkyl groups can be optionally substituted as described herein. An alkyl group is generally not substituted with another alkyl group, an alkenyl group, or an alkynyl group.

As used herein, “alkenyl” refers to a straight-chain or branched alkyl group having one or more carbon-carbon double bonds. Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In various embodiments, an alkenyl group can have 2 to 40 carbon atoms (i.e., C2-40 alkenyl group), for example, 2 to 20 carbon atoms (i.e., C2-20 alkenyl group). In certain embodiments, alkenyl groups can be substituted as described herein. An alkenyl group is generally not substituted with another alkenyl group, an alkyl group, or an alkynyl group.

As used herein, “cycloalkyl” by itself or as part of another substituent means, unless otherwise stated, a monocyclic hydrocarbon having between 3-12 carbon atoms in the ring system and includes hydrogen, straight chain, branched chain, and/or cyclic substituents. Exemplary cycloalkyls include cyclopropyl, cyclobutyl, cyclopentyl, cyclohexyl, cycloheptyl, and the like.

As used herein, “heteroatom” refers to an atom of any element other than carbon or hydrogen and includes, for example, nitrogen, oxygen, silicon, sulfur, phosphorus, and selenium.

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulfur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or heterocycloalkyl rings. An aryl group can have 6 to 24 carbon atoms in its ring system (e.g., C6-24 aryl group), which can include multiple fused rings. In certain embodiments, a polycyclic aryl group can have 8 to 24 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic), pentacenyl (pentacyclic), and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. In certain embodiments, aryl groups can be optionally substituted.

The term “aralkyl” refers to an alkyl group substituted with an aryl group.

The term “optionally substituted” refers to a chemical group, such as alkyl, cycloalkyl aryl, and the like, wherein one or more hydrogen may be replaced with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” and “sulfone” is art-recognized and refers to —SO₂—. “Halide” designates the corresponding anion of the halogens.

As used herein, the term “substantially pure” in connection with a sample of a compound described herein means the sample contains at least 60% by weight of the compound. In certain embodiments, the sample contains at least 70% by weight of the compound; at least 75% by weight of the compound; at least 80% by weight of the compound; at least 85% by weight of the compound; at least 90% by weight of the compound; at least 95% by weight of the compound; or at least 98% by weight of the compound.

The symbol “ [symbol] ” or “ [symbol] ” or “ [symbol] ” or “ [symbol] ” in a chemical structure represents a position from where the specified chemical structure is bonded to another chemical structure.

The present disclosure provides a rhenium(V) alkylidyne complex having the chemical Formula 1:

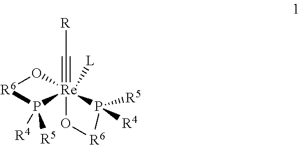

wherein L is H₂O, an ether, a ketone, a furan, a thioether, a thiophene, a tertiary amine, or a metal salt selected from the group consisting of a metal halide, a metal alkoxide, a metal thiolate and a metal amide;
R is alkyl, aryl, thiophene, or CH₂Ari, wherein Ar¹ is aryl;
each of R⁴ and R⁵ is independently alkyl, cycloalkyl or aryl; and
R⁶ for each instance is independently alkyl, alkenyl or aryl.

In the interest of clarity, the rhenium(V) alkylidyne complex described herein is shown as a single isomer. However, the present disclosure contemplates all isomeric forms of the rhenium(V) alkylidyne complex, including structural isomers, geometric isomers, and stereoisomers. The rhenium(V) alkylidyne complex described herein can exist in enantiopure pure form (e.g., having enantiomeric excess of 50-100%, 50-99.9%, 60-99.9%, 70-99.9%, 80-99.9%, 90-99.9%, 95-99.9%, 97-99.9%, or 99-99.9%) or as a racemic mixture.

In certain embodiments, the rhenium(V) alkylidyne complex is substantially pure.

In certain embodiments, L is H₂O, a 2-alkyl substituted furan, such as 2-methyl furan, 2-ethyl furan, 2-propyl furan, or 2-isopropyl furan, a 2-alkyl substituted thiophene, such as 2-methyl thiophene, 2-ethyl thiophene, 2-propyl thiophene, or 2-isopropyl thiophene, a ketone, such as acetone, 2-butanone, or methyl isopropyl ketone, a halide, such as chloride, bromide, or iodide, a thiolate, such as —S—C₁-C₆alkyl, —S—C₁-C₅ alkyl, —S—C₁-C₄ alkyl, —S—C₁-C₃ alkyl, or —S—C₁-C₂ alkyl, a tertiary amine, such as N(R⁷)₃, wherein R⁷ for each instance is independently selected from C₁-C₆ alkyl, C₁-C₅ alkyl, C₁-C₄ alkyl, C₁-C₃ alkyl, and C₁-C₂ alkyl, or two instances of R⁷ taken together with the nitrogen to which they are covalently bonded form a 3-6, 3-5, 3-4, or 4-6 membered cycloalkyl amine, a dialkylamido, such as —N(R⁷)₂, wherein R⁷ for each instance is independently selected from C₁-C₆ alkyl, C₁-C₅ alkyl, C₁-C₄ alkyl, C₁-C₃ alkyl, and C₁-C₂ alkyl, or two instances of R⁷ taken together with the nitrogen to which they are covalently bonded form a 3-6, 3-5, 3-4, or 4-6 membered cycloalkyl amine, or an dialkyl ether or a dialkyl thioether represented by R⁷YR⁷, wherein Y is O or S and R⁷ for each instance is independently selected from C₁-C₆ alkyl, C₁-C₅ alkyl, C₁-C₄ alkyl, C₁-C₃ alkyl, and C₁-C₂ alkyl, or both instances of R⁷ taken together with the oxygen to which they are covalently bonded form a 3-6, 3-5, 3-4, or 4-6 membered cycloalkyl ether. Exemplary alkyl ethers include, but are not limited to dimethyl ether, diethyl ether, diisopropyl ether, tert-butyl methyl ether, 2-methyl tetrahydrofuran, 2-ethyl tetrahydrofuran, tetrahydropyran, and the like. In certain embodiments, L is H₂O, diethyl ether, tetrahydrofuran, or tetrahydropyran.

In instances in which L is a metal salt or a metal complex, L can be bound (e.g., via a dative bond) to a bridging halide, alkoxide, thiolate, or amide, which is bound to one or more metal cations as illustrated in the exemplary embodiment in which L is MgCl₂, ZnCl₂, RuCl₂(C₆H₆), and PdCl₂(COD) below:

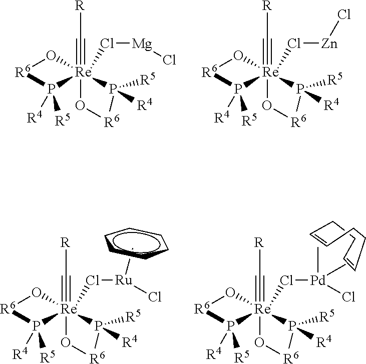

wherein each of R, R⁴, R⁵, and R⁶ is independently described herein. In certain embodiments, the metal salt is represented by represented by the formula (A^{t+})_{U}(B^{u-})_{T}, wherein A represents the metal cation selected from the group consisting of Group 1-14 elements, B represents an anion selected from the group consisting of halide, R⁷O⁻, R⁷S⁻, and the (R⁷)₂N⁻, wherein R⁷ for each instance is independently selected from C₁-C₆ alkyl, C₁-C₅ alkyl, C₁-C₄ alkyl, C₁-C₃ alkyl, and C₁-C₂ alkyl, or two instances of R⁷ taken together with the nitrogen to which they are covalently bonded form a 3-6, 3-5, 3-4, or 4-6 membered cycloamide, u represents the charge of the anion, U is equal to the absolute value of the charge of the anion and T is equal to the absolute value of the charge of the metal cation. Exemplary metal cations include, but are not limited to, Li, Na, K, Rb Cs, Mg, Ca, Sr, Ba, Zr, Ti, Cr, Mn, Fe, Ru, Os, Co, Rh, Ir, Ni, Pd, Pt, Cu, Ag, Au, Zn, Hg, Al, Ga, In, Sn, and Pb.

In certain embodiments, R is phenyl, 2-thiophene, or CH₂Ari, wherein Ar¹ is phenyl and o-bromophenyl.

In certain embodiments, each of R⁴ and R⁵ is independently cyclohexyl or phenyl optionally substituted with one or more substituents selected from alkyl, CH₃, OCH₃, F, and CF₃. Each phenyl can be independently substituted with 0, 1, or 2 substituents. In certain embodiments, each of R⁴ and R⁵ is independently a moiety selected from the group consisting of phenyl, 4-fluorophenyl, 2,4-difluorophenyl, 4-trifluoromethylphenyl, 2,4-bis(trifluoromethyl)phenyl, 2,4-dimethylphenyl, and 2,4-dimethoxyphenyl.

In certain embodiments, R⁶ is phenyl optionally substituted with alkyl or trifluoromethyl. In certain embodiments, R⁶ is a moiety selected from the group consisting of:

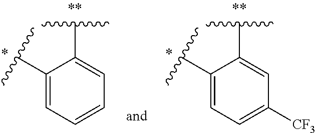 [SMILES: *c1ccccc1* ; *c1ccc(C(F)(F)F)cc1*]

wherein * represents the site of the covalent bond to oxygen and ** represents the site of the covalent bond to phosphorous.

In certain embodiments, the rhenium(V) alkylidyne complex has the Formula 3

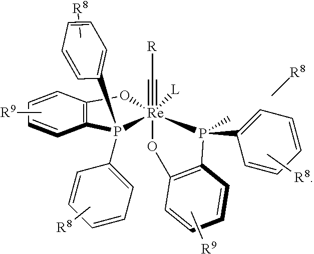

wherein R⁸ is alkyl; R⁹ is alkyl or a hetero atom-containing group selected from the group consisting of F, CF₃, and OMe; L is H₂O, tetrahydrofuran, tetrahydropyran, or Et₂O; and R is benzyl.

In certain embodiments, the rhenium(V) alkylidyne complex is selected from the group consisting of:

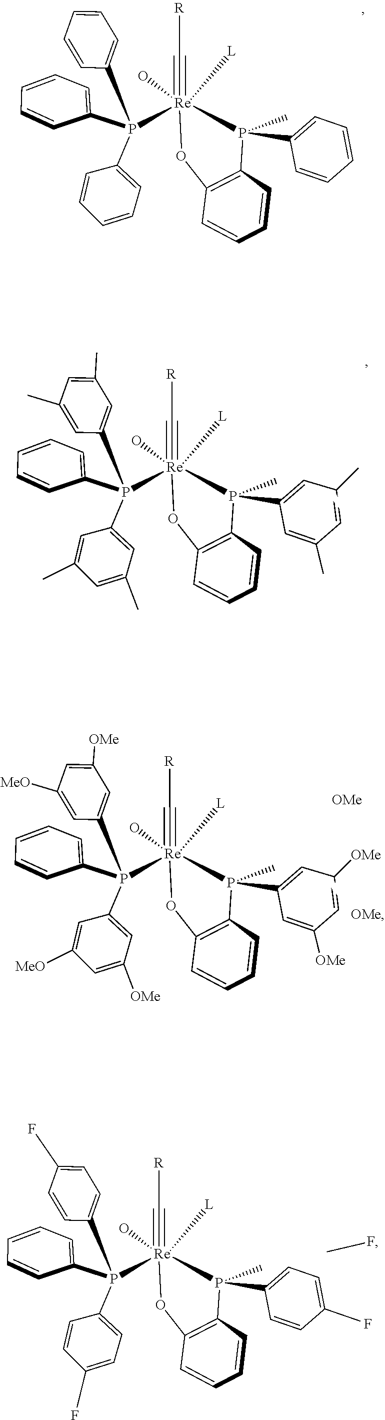

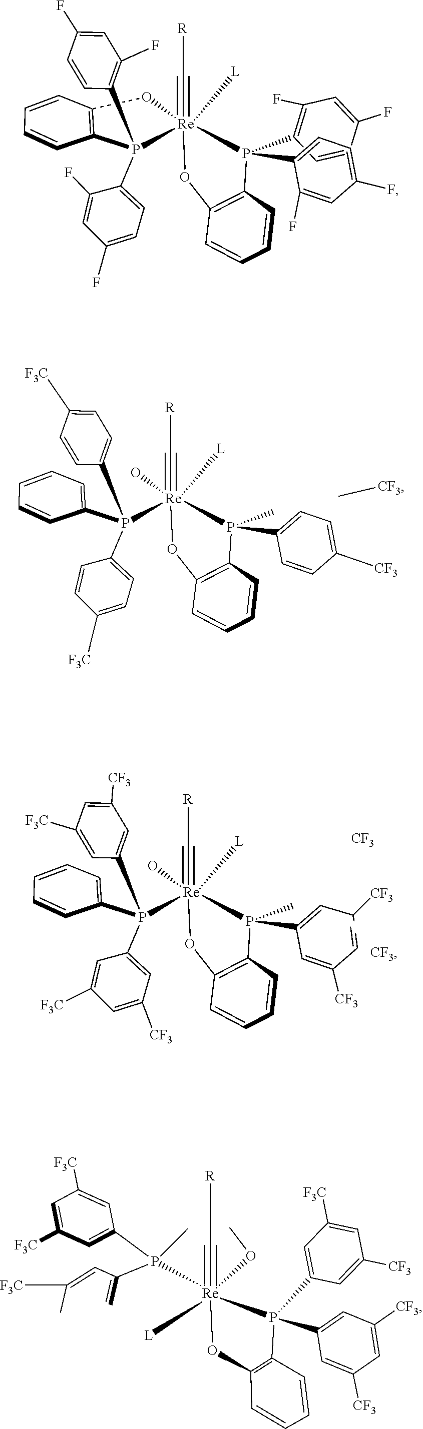

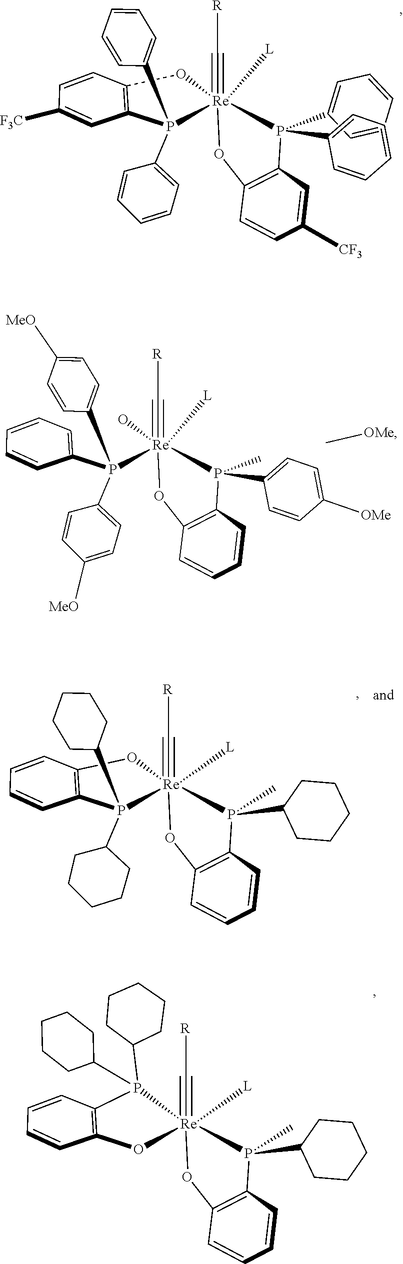

wherein R is alkyl, aryl, 2-thiophene, or CH₂Ar¹, wherein Ar¹ is phenyl or o-bromophenyl; and L is H₂O.

In certain embodiments, the rhenium (V) alkylidyne complex has the structure:

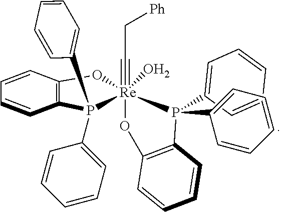

The rhenium (V) alkylidyne complex of Formula 1 herein can be synthesized using methods well known to those skilled in the art. In certain embodiments, the rhenium (V) alkylidyne complex of Formula 1 is prepared by ligand substitution reactions of rhenium (V) alkylidyne complexes with phosphino phenols or phosphino alcohols. In certain embodiments, the rhenium (V) alkylidyne complexes described herein are prepared as illustrated in Scheme 1:

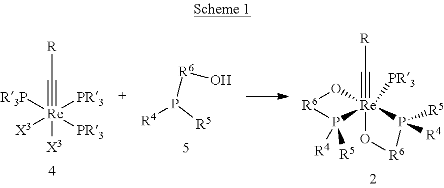

wherein each of R⁴, R⁵, and R⁶ are each as defined in any embodiment described herein; X³ is Cl; PR′₃ is PMePh₂ or PMe₂Ph; and R is alkyl, aryl, thiophene, or CH₂ArO, wherein Ar¹ is aryl. The rhenium (V) alkylidyne complex of Formula 2 can be prepared as described by W. Bai et al. in Organometallics 2016, 35, 3808-3815, Organometallics 2018, 37, 559-569 or G. He et al. in New J. Chem. 2013, 37, 1823-1832.

Generally, the synthesis reactions can be carried out at 100° C. in toluene under N₂. Addition of excess (2-5 equiv.) organic base, such as triethyl amine can accelerate the transformation. The reaction will generally be finished within hours and after the reaction, insoluble salts can be removed by filtration and PR′₃ can be removed by washing with hexane, ether, methanol, or their mixtures.

The rhenium (V) alkylidyne complexes described herein can be synthesized by ligand exchange reactions of existing rhenium (V) alkylidyne complexes with a neutral electron donor ligand. For example, the reaction formula can be written as shown in Scheme 2:

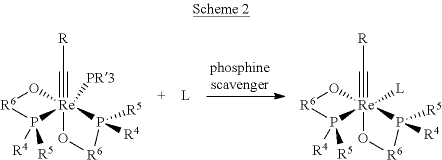

wherein each of L, R, R′, R⁴, R⁵, and R⁶ is independently as defined in any embodiment described herein; and the phosphine scavenger is selected from the group consisting of [(p-cymene]RuCl₂]₂, [(p-cymene]OsCl₂]₂, [RhCl(COD)]₂, IrCl(COD)]₂, PdCl₂(COD), CuCl, and CuI.

Generally, the synthesis reactions can be carried out at 80-140° C. or 100-140° C. in toluene under N₂. The amount of phosphine scavenger added can be 1 equiv. The reaction can be finished within hours. Advantageously, the products can be air and moisture stable in in solution. After the reaction, the byproduct phosphine complexes can be readily removed by washing with hexane, ether, methanol or their mixtures. More particularly, the product can be purified by diethyl ether extraction followed by recrystallization from diethyl ether only when the product can well-dissolved in diethyl ether.

The present disclosure provides a method of preparing the rhenium (V) alkylidyne complex of Formula 1, the method comprising: contacting a catalyst precursor of Formula 2:

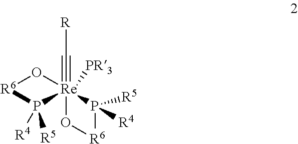

wherein R is alkyl, aryl, thiophene, or CH₂Ari, wherein Ar¹ is aryl;
PR′₃ is PMePh₂ or PMe₂Ph;
each of R⁴ and R⁵ is independently aryl, alkyl, or cycloalkyl; and
R⁶ for each instance is independently alkyl, alkenyl or aryl;
with a ligand selected from the group consisting of H₂O and an alkyl ether; and a phosphine scavenger selected from phosphine scavenger is selected from [(p-cymene]RuCl₂]₂, [(p-cymene]OsCl₂]₂, [RhCl(COD)]₂, IrCl(COD)]₂, PdCl₂(COD), CuCl, or CuI thereby forming the rhenium (V) alkylidyne complex of Formula 1.

In certain embodiments, R is CH₂ArO, wherein Ar¹ is phenyl or o-bromophenyl.

In certain embodiments, each of R⁴ and R⁵ is independently cyclohexyl, phenyl optionally substituted with one or more substituents selected from alkyl, CH₃, OCH₃, F, and CF₃. Each phenyl can be independently substituted with 0, 1, or 2 substituents. In certain embodiments, each of R⁴ and R⁵ is independently a moiety selected from the group consisting of phenyl, 4-fluorophenyl, 2,4-difluorophenyl, 4-trifluoromethylphenyl, 2,4-bis(trifluoromethyl)phenyl, 2,4-dimethylphenyl, and 2,4-dimethoxyphenyl.

In certain embodiments, R⁶ is phenyl optionally substituted with alkyl or trifluoromethyl. In certain embodiments, R⁶ is a moiety selected from the group consisting of:

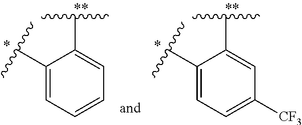 [SMILES: *c1ccccc1* ; *c1ccc(C(F)(F)F)cc1*]

wherein * represents the site of the covalent bond to oxygen and ** represent the site of the covalent bond to phosphorous.

The method of preparing the rhenium (V) alkylidyne complex of Formula 1 can further comprise the step of contacting a compound of Formula 4:

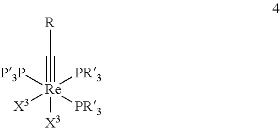

wherein X³ is Cl; PR₃ is PMePh₂ or PMe₂Ph; R is alkyl, aryl, thiophene, or CH₂ArO, wherein Ar¹ is aryl; with a compound of Formula 5:

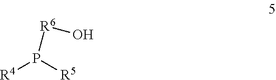

wherein each of R⁴ and R⁵ is independently alkyl, cycloalkyl, or aryl; and R⁶ is alkyl, alkenyl or aryl thereby forming the catalyst precursor of Formula 2.

Also provided herein is a method of method for performing a metathesis reaction, the method comprising: contacting a rhenium(V) alkylidyne complex described herein with at least one alkyne substrate thereby forming an alkyne metathesis product.

The rhenium(V) alkylidyne complex exhibits a high degree of functional group compatibility. Accordingly, the types of alkyne substrates useful in the methods described herein are not particularly limited. In certain embodiments, the alkyne substrate is selected from the group consisting of an acyclic alkyne, a cyclic alkyne, an acyclic diyne, and a cyclic diyne. In certain embodiments, the alkyne substrate comprises one or more functional groups selected from the group consisting of: alkyl, aralkyl, cycloalkyl, heterocycloalkyl, aryl, heteroaryl, a perhaloalkyl, a nitro, a nitrile, a halide (e.g. F, Cl, Br, or I), a hydroxyl, an ether, a thioether, a tertiary amine, an aldehyde, a ketone, an ester, an amide, a carbonate, a phosphite, phosphonate, a phosphate, a carbamate, a urea, and a sulfonamide.

The rhenium(V) alkylidyne complex can be present in the metathesis reaction in a catalytic, stoichiometric, or an excess relative to the alkyne substrate. In certain embodiments, the rhenium(V) alkylidyne complex is present at 0.1-10 mol %, 0.5-10 mol %, 1-10 mol %, 3-7 mol %, 4-6 mol %, or 1-5 mol %, relative to the alkyne substrate. In certain embodiments, the rhenium(V) alkylidyne complex is present at about 5 mol % relative to the alkyne substrate.

The metathesis reaction catalyzed using the rhenium(V) alkylidyne complex described herein can be conducted in a non-polar organic solvent, a polar organic solvent, or a mixture thereof. In certain embodiments, the solvent is an aromatic solvent, such as benzene, toluene, xylene, or a mixture thereof.

The rhenium(V) alkylidyne complex described herein is a highly active metathesis catalyst. Consequently, the metathesis reaction catalyzed using the rhenium(V) alkylidyne complex described herein can be conducted at any temperature. For example, the metathesis reaction can be conducted at a temperature between −40° C.-200° C., −40° C.-180° C., −40° C.-160° C., −40° C.-140° C., −40° C.-120° C., −40° C.-100° C., −20° C.-100° C., 0° C.-100° C., 10° C.-100° C., 20° C.-100° C., 20° C.-90° C., 20° C.-80° C., 30° C.-80° C., 40° C.-80° C., 50° C.-70° C., 55° C.-65° C., 20° C.-70° C., 20° C.-60° C., 20° C.-50° C., 20° C.-40° C., 20° C.-30° C., 25° C.-30° C., or 20° C.-25° C. In certain embodiments, the metathesis reaction catalyzed using the rhenium(V) alkylidyne complex described herein is conducted at about 25° C. or about 60° C.

The metathesis reaction using the rhenium(V) alkylidyne complex described herein can be conducted for any length of time. The selection of the appropriate reaction time is well within the skill of a person of ordinary skill in the art. In certain embodiments, the metathesis reaction using the rhenium(V) alkylidyne complex described herein is conducted for 1-20 hours, 5-20 hours, 10-20 hours, 15-20 hours, 1-15 hours, 1-10 hours, 1-5 hours, or 2-4 hours. In certain embodiments, the metathesis reaction using the rhenium(V) alkylidyne complex described herein is conducted for about 1 hour, about 3 hours, or about 16 hours.

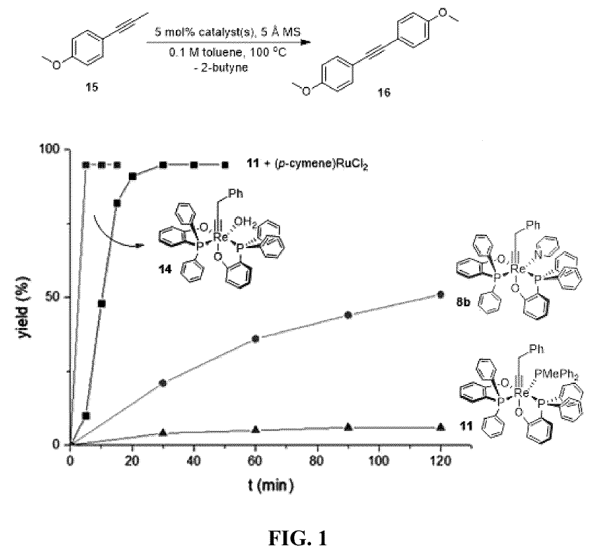

FIG. 1 shows the results of homometathesis of a model alkyne using rhenium(V) alkylidyne complexes described herein. The results show that the rhenium (V) alkylidyne complexes described herein are more active than pyridine and phosphine substituted analogs.

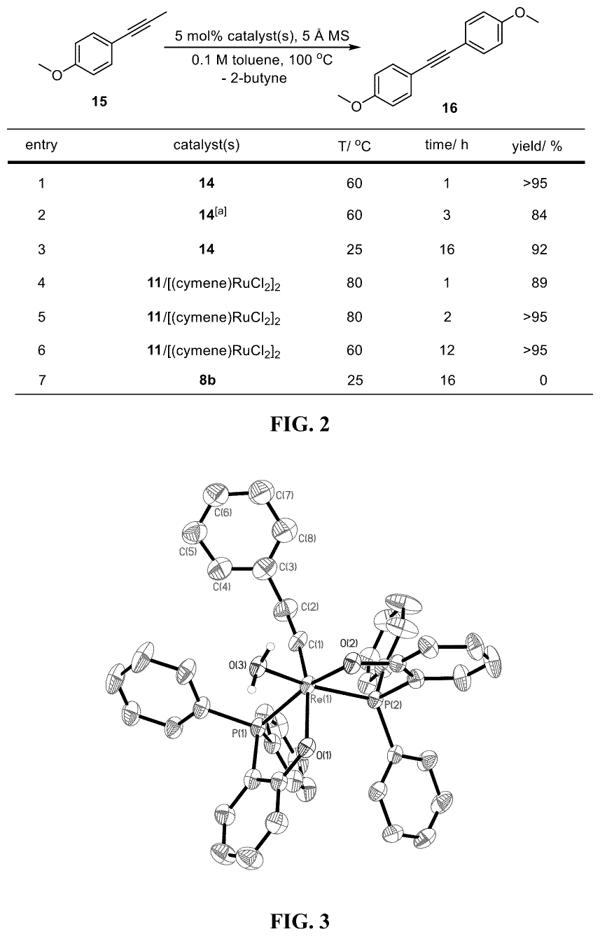

FIG. 2 shows a table presenting the results of homometathesis of a model alkyne using the rhenium(V) alkylidyne complexes described herein. Advantageously, the rhenium(V) alkylidyne complexes described herein produce the desired alkyne metathesis product in high yield at 25-80° C.

The present disclosure also provides a method of method for performing a metathesis reaction, the method comprising: contacting a catalyst precursor of Formula 2, a phosphine scavenger, and at least one alkyne substrate thereby forming an alkyne metathesis product, wherein the catalyst precursor of Formula 2 has the structure:

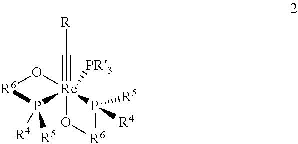

wherein R is alkyl, aryl, thiophene, or CH₂Ari, wherein Ar¹ is aryl;
PR′₃ is PMePh₂ or PMe₂Ph;
each of R⁴ and R⁵ is independently alkyl, cycloalkyl, or aryl; and
R⁶ for each instance is independently alkyl, alkenyl or aryl; and
the phosphine scavenger is selected from [(p-cymene]RuCl₂]₂, [(p-cymene]OsCl₂]₂, [RhCl(COD)]₂, IrCl(COD)]₂, PdCl₂(COD), CuCl, or CuI.

In certain embodiments of the catalyst precursor of Formula 2, R is phenyl, 2-thiophene, or CH₂ArO, wherein Ar¹ is phenyl and o-bromophenyl.

In certain embodiments of the catalyst precursor of Formula 2, each of R⁴ and R⁵ is independently cyclohexyl or phenyl optionally substituted with one or more substituents selected from alkyl, CH₃, OCH₃, F, and CF₃. Each phenyl can be independently substituted with 0, 1, or 2 substituents. In certain embodiments, each of R⁴ and R⁵ is independently a moiety selected from the group consisting of phenyl, 4-fluorophenyl, 2,4-difluorophenyl, 4-trifluoromethylphenyl, 2,4-bis(trifluoromethyl)phenyl, 2,4-dimethylphenyl, and 2,4-dimethoxyphenyl.

In certain embodiments of the catalyst precursor of Formula 2, R⁶ is phenyl optionally substituted with alkyl or trifluoromethyl. In certain embodiments, R⁶ is a moiety selected from the group consisting of:

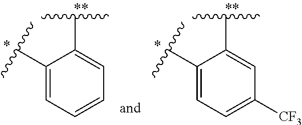 [SMILES: *c1ccccc1* ; *c1ccc(C(F)(F)F)cc1*]

wherein * represents the site of the covalent bond to oxygen and ** represents the site of the covalent bond to phosphorous.

In certain embodiments, the catalyst precursor has the Formula 6:

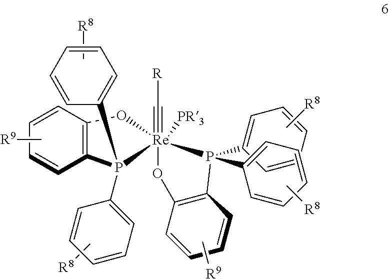

wherein R s is alkyl; R⁹ is alkyl or a hetero atom-containing group selected from the group consisting of F, CF₃, and OMe; PR′3 is PMePh₂ or PMe₂Ph; and R is benzyl.

In certain embodiments, the catalyst precursor is selected from the group consisting of:

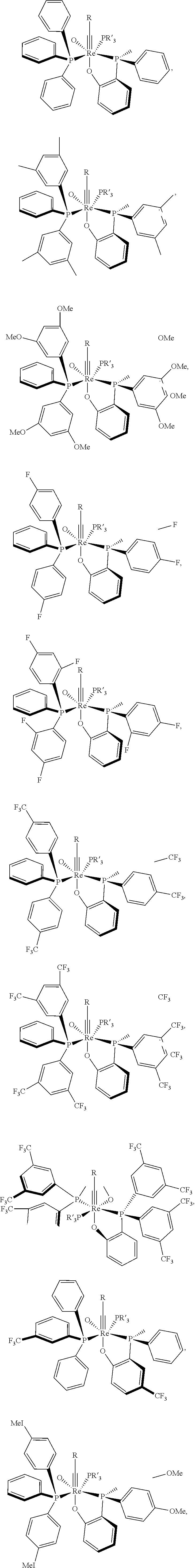

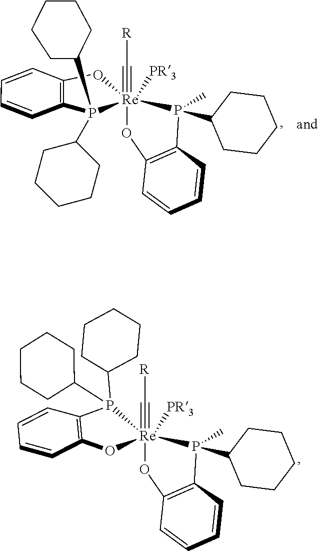

wherein R is alkyl, aryl, 2-thiophene, or CH₂Ar¹, wherein Ar¹ is phenyl or o-bromophenyl; and PR′3 is PMePh₂ or PMe₂Ph.

In certain embodiments, the catalyst precursor has the structure:

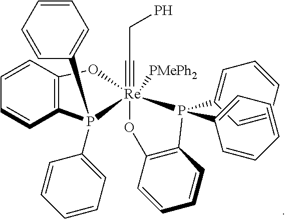

The metathesis catalyst system comprising the catalyst precursor of Formula 2 and phosphine scavenger exhibits a high degree of functional group compatibility. Accordingly, the types of alkyne substrates useful in the methods described herein are not particularly limited. In certain embodiments, the alkyne substrate is selected from the group consisting of an acyclic alkyne, a cyclic alkyne, an acyclic diyne, and a cyclic diyne. In certain embodiments, the alkyne substrate comprises one or more functional groups selected from the group consisting of: alkyl, aralkyl, cycloalkyl, heterocycloalkyl, aryl, heteroaryl, a perhaloalkyl, a nitro, a nitrile, a halide (e.g., F, Cl, Br, or I), a hydroxyl, an ether, a thioether, a tertiary amine, an aldehyde, a ketone, an ester, an amide, a carbonate, a phosphite, phosphonate, a phosphate, a carbamate, a urea, and a sulfonamide.

The metathesis reaction catalyzed using metathesis catalyst system comprising the catalyst precursor of Formula 2 and phosphine scavenger described herein can be conducted in a non-polar organic solvent, a polar organic solvent, or a mixture thereof. In certain embodiments, the solvent is an aromatic solvent, such as benzene, toluene, xylene, or a mixture thereof.

The catalyst precursor of Formula 2 and the phosphine scavenger can be present in any mol ratio. In certain embodiments, the catalyst precursor of Formula 2 and the phosphine scavenger are present in a mol ratio of 1:1 to 1:2, 1:1 to 1:1.5, 1:1 to 1:1.25, 1:1 to 1:1.1, 1:1 to 1:1.05, 1:0.1 to 1:0.9, 1:0.2 to 1:0.8, 1:0.3 to 1:0.7, 1:0.4 to 1:0.6, or 1:0.45 to 1:0.55, respectively. In certain embodiments, the catalyst precursor of Formula 2 and the phosphine scavenger are present in a mol ratio of about 1 to about 1 or about 1 to about 0.5, respectively.

The metathesis catalyst system comprising the catalyst precursor of Formula 2 described herein can be present in the metathesis reaction in a catalytic, stoichiometric, or an excess relative to the alkyne substrate. In certain embodiments, the catalyst precursor of Formula 2 is present at 0.1-10 mol %, 0.5-10 mol %, 1-10 mol %, 3-7 mol %, 4-6 mol %, or 1-5 mol %, relative to the alkyne substrate. In certain embodiments, the rhenium(V) alkylidyne complex is present at about 5 mol % relative to the alkyne substrate.

The metathesis catalyst system comprising the catalyst precursor of Formula 2 and phosphine scavenger described herein is a highly active metathesis catalyst system. Consequently, the metathesis reaction catalyzed using the catalyst system comprising the catalyst precursor of Formula 2 and phosphine scavenger described herein can be conducted at any temperature. For example, the metathesis reaction can be conducted at a temperature between 25° C.-200° C., 40° C.-150° C., 40° C.-150° C., 40° C.-100° C., 50° C.-90° C., 60° C.-80° C., 70° C.-80° C., 60° C.-70° C., 40° C.-80° C., 50° C.-70° C., 55° C.-65° C., 60° C.-100° C., 70° C.-90° C., or 75° C.-85° C. In certain embodiments, the metathesis reaction catalyzed using the catalyst system comprising the catalyst precursor of Formula 2 and phosphine scavenger described herein is conducted at about 60° C. or about 80° C.

The metathesis reaction using catalyst system comprising the catalyst precursor of Formula 2 and phosphine scavenger described herein can be conducted for any length of time. The selection of the appropriate reaction time is well within the skill of a person of ordinary skill in the art. In certain embodiments, the metathesis reaction using the catalyst system comprising the catalyst precursor of Formula 2 and phosphine scavenger described herein is conducted for 1-20 hours, 5-20 hours, 10-20 hours, 15-20 hours, 1-15 hours, 1-10 hours, 1-5 hours, or 2-4 hours. In certain embodiments, the metathesis reaction using the rhenium(V) alkylidyne complex described herein is conducted for about 1 hour, about 3 hours, or about 16 hours.

### EXAMPLES

### General Considerations.

All manipulations were carried out under a nitrogen atmosphere using standard Schlenk techniques unless stated otherwise. Solvents were freshly distilled under nitrogen from sodium benzophenone (hexane, diethyl ether, tetrahydrofuran, toluene), or CaH₂ (dichloromethane). Methanol and ethanol were bubbled with N₂ for about 20 min before use. Deuterated solvents were dried over CaH₂ (CD₂Cl₂, CDCl₃) or sodium benzophenone (C₆D₆), distilled or vacuum transferred under nitrogen, degassed by three freeze-pump-thaw cycles and stored in a sealed tube with 4 Å molecular sieves. Powdered 5 Å molecular sieves (5 Å MS) was purchased from Sigma-Aldrich and activated prior to use either by heating at 150° C. under vacuum for about 24 h or heating with a heat gun (at 450° C.) under vacuum for about 5 min. ¹H, ¹³C{¹H}, and ³¹P{¹H}NMR spectra were collected on a Bruker-400 spectrometer. ¹H and ¹³C{¹H}NMR shifts are reported in ppm and are relative to the solvent signal (¹H NMR, CDCl₃ at 7.26 ppm, CD₂Cl₂ at 5.32 ppm, C₆D₆ at 7.16 ppm; ¹³C{¹H}NMR, CDCl₃ at 77.16 ppm, CD₂Cl₂ at 53.84 ppm, C₆D₆ at 128.06 ppm). ³¹P{¹H}chemical shifts are relative to 85% H₃PO₄. FT-IR spectra were recorded on a Bruker ALPHA spectrometer with an ATR attachment, and selected peaks are reported in cm⁻¹. HRMS were recorded by using a chemical ionization (CI) or electrospray ionization (ESI) mass spectrometer. Elemental analysis was performed by MEDAC Ltd (Egham, UK).

### Example 1—Large Scale Synthesis of Re(≡CCH₂Ph)(^{Ph}PO)₂(PMePh₂) (Complex 11)

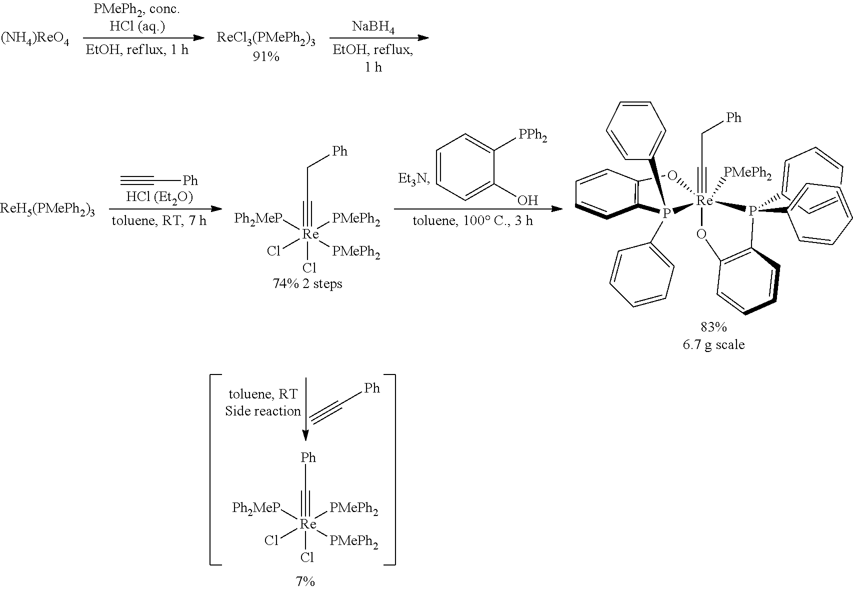

mer-ReCl₃(PMePh₂)₃.

To a 250 mL 3-neck flask equipped with a gas inlet, a condenser and a stir bar were added (NH₄)ReO₄ (6.50 g, 24.2 mmol), concentrated hydrochloric acid (aq., 18.4 mL) and 100 mL of degassed ethanol. The mixture was stirred for 30 min until the solid was fully dissolved, to which was added PMePh₂ (25.00 g, 125.0 mmol). Under vigorous stirring, the mixture was refluxed for 1 hour until a yellow suspension was obtained. Note: Normally, the powdered product will precipitate all of a sudden, accompanied with the formation of oily phosphine oxide. The vigorous stirring is crucial to avoid the formation of deep-green chunks, which prevent the reaction to go completion. The mixture was cooled to room temperature and the air-stable yellow powder was collected by filtration, washed with ethanol (30 ml×3) and dried under vacuum. Yield: 20.07 g, 91%. 1H NMR (400 MHz, CDCl₃) δ 13.90 (d, J=7.7 Hz, 8H), 9.83 (d, J=7.6 Hz, 4H), 9.42 (t, J=7.5 Hz, 2H), 8.86 (t, J=7.5 Hz, 4H), 8.58 (t, J=7.6 Hz, 8H), 7.89 (t, J=7.5 Hz, 4H), −0.69 (s, 6H), −2.12 (s, 3H).

Re(≡CCH₂Ph)Cl₂(PMePh₂)₃.

To a 250 mL 3-neck flask equipped with a gas inlet, a condenser and a stir bar were added ReCl₃(PMePh₂)₃ (9.5 g, 10.6 mmol), NaBH₄ (5.25 g, 140 mmol) and 130 mL of degassed ethanol. The mixture was vigorously stirred at room temperature for 20 min and then heated to reflux for about 1 hour until gas evolution ceased. After cooling down to room temperature, the solvent was completely evaporated under vacuum to give a pale red solid. The red ReH₅(PMePh₂)₃ was extracted out with toluene (45 mL×3) from the mixture and the white inorganic salts were filtered off and discarded. The combined filtrates were collected in a 250 mL Schlenk flask charged with a stir bar, to which was added phenylacetylene (2.94 g, 28.7 mmol, ca. 3.15 mL). At room temperature, 28.7 mL of 1 M ethereal solution of HCl was added dropwise to the stirred solution over 1 h. During the course of addition, the solution color changed gradually from red to brown, followed by gentle gas evolution. After the addition, the reaction mixture was further stirred at room temperature for 6-7 hours until a large amount of yellow precipitant was formed. Note: Prolonged reaction time may cause the increase of the phenyl by-product Re(═CPh)Cl₂(PMePh₂)₃, which was generated from alkyne metathesis of Re(═CCH₂Ph)Cl₂(PMePh₂)₃ with excess phenylacetylene. The volume of the solution was reduced to one-half under vacuum and 120 mL of hexane was added to the solution to precipitate more solid. The solid was collected by cannula filtration, sequentially washed with hexane (60 mL×2), diethyl ether (60 mL×3) as well as methanol (16 mL×3) and dried under vacuum to afford the desired product Re(≡CCH₂Ph)Cl₂(PMePh₂)₃ as a yellow solid. Yield: 7.42 g, 74% for the two-step synthesis from ReCl₃(PMePh₂)₃.

A small amount of the side product, Re(≡CPh)Cl₂(PMePh₂)₃, could be isolated and purified by the following procedure: The toluene/hexane filtrate and Et₂O extracts were combined and evaporated to dryness to give an oily residue, which was washed with methanol (15 mL×3). The resulting orange solid was extracted with toluene (10 mL×3). The extract was filtered by cannula filtration into a long Schlenk tube and layered with hexane. After 2 weeks, red crystals (benzylidyne) together with orange crystals (benzyl alkylidyne) were deposited on the wall of the Schlenk tube. The red crystals were picked up by hands using a spatula. Repeating this procedure for two times gave pure Re(≡CPh)Cl₂(PMePh₂)₃ as red crystals. Yield: 705 mg (7% for 2 steps).

Characterization data of Re(≡CCH₂Ph)Cl₂(PMePh₂)₃: ¹H NMR (400 MHz, CDCl₃) δ 7.54-7.45 (m, 4H), 7.40-7.31 (m, 4H), 7.31-7.10 (m, 15H), 7.10-6.94 (m, 12H), 2.68 (q, J=3.6 Hz, 2H), 2.14 (t, J=4.0 Hz, 6H), 1.80 (d, J=8.8 Hz, 3H). ³¹P{¹H}NMR (162 MHz, CDCl₃) δ −7.19 (t, J=11.4 Hz, 1P), −9.59 (d, J=11.5 Hz, 2P).

Characterization data of Re(≡CPh)Cl₂(PMePh₂)₃: ¹H NMR (400 MHz, CDCl₃) δ 7.45-6.85 (m, 35H), 2.20 (t, J=4.0 Hz, 6H), 1.81 (d, J=8.6 Hz, 3H). ³¹P{¹H}NMR (162 MHz, CDCl₃) δ −3.41 (t, J=10.9 Hz, 1 P), −11.18 (d, J=11.6 Hz, 2P).

Re(≡CCH₂Ph)(^{Ph}PO)₂(PMePh₂). To a 250 mL Schlenk flask equipped with a stir bar were added Re(≡CCH₂Ph)Cl₂(PMePh₂)₃(7.42 g, 7.7 mmol), (2-hydroxyphenyl)diphenylphosphine (4.50 g, 16.2 mmol), 80 mL of toluene and Et₃N (2.34 g, ca. 3.2 mL, 23.2 mmol). The mixture was stirred at 100° C. for 3 hours. After cooling down to room temperature, the precipitate (triethylamine hydrochloride) was filtered off and discarded. The filtrate was evaporated under vacuum to give an orange residue which was washed with hexane (50 mL×2) and methanol (20 mL×3) and dried under vacuum to afford the desired product as a yellow solid. Yield: 6.66 g, 83%.

¹H NMR (400.1 MHz, CDCl₃): δ 7.36-7.23 (m, 5H), 7.21-7.10 (m, 15H), 7.05-6.90 (m, 14H), 6.84-6.76 (m, 5H), 6.59-6.42 (m, 4H), 2.80 (dq, J=19.8, 3.2 Hz, 1H), 2.56 (dq, J=19.6, 2.8 Hz, 1H), 1.31 (d, J=8.5 Hz, 3H). ¹H NMR (400 MHz, C₆D₆) 6 7.81-7.71 (m, 2H), 7.65-7.50 (m, 4H), 7.47-7.33 (m, 3H), 7.28-7.18 (m, 2H), 7.15-6.68 (m, 30H), 6.60-6.45 (m, 2H), 2.79 (dq, J=19.7, 3.5 Hz, 1H), 2.55 (dq, J=19.9, 3.6 Hz, 1H), 1.27 (dd, J=8.5, 1.0 Hz, 3H). ³¹P{¹H}NMR (162.0 MHz, CDCl₃): δ 37.28 (br s, 1P), 27.00 (br d, J=225.3 Hz, 1P), 3.39 (br d, J=224.0 Hz, 1P). ³¹P{¹H}NMR (162 MHz, C₆D₆) 6 38.92 (br s, 1P), 25.88 (dd, J=222.9, 5.6 Hz, 1P), 4.99 (d, J=223.9, 1P).

### Example 2—Synthesis of the aqua complex Re(≡CCH₂Ph)(^{Ph}PO)₂(H₂O)

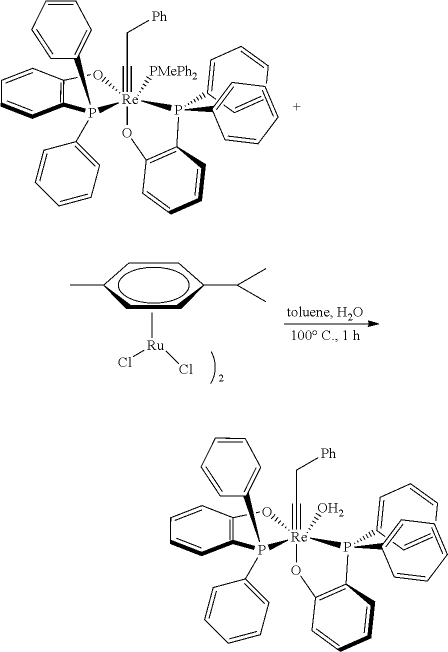

To a Schlenk flask equipped with a stir bar were added Re(≡CCH₂Ph)(^{Ph}PO)₂(PMePh₂) (1 g, 0.958 mmol), [(p-cymene)RuCl₂]₂ (391 mg, 1.28 mmol), 28 mL of dry toluene. The resulting mixture was stirred at 100° C. with vigorous stirring for 1 hour. The hot solution was filtered to another Schlenk flask to remove a trace amount of black impurities originated from [(p-cymene)RuCl₂]₂. To the filtrate was added 140 μL of distilled water (7.78 mmol) and the mixture was stirred at 100° C. for another 1 h. After the reaction, the mixture was cooled to 0° C. to give a yellow crystalline solid, which was collected by filtration, washed with toluene (15 mL×4) and dried under vacuum. Yield: 744 mg, 90%. ¹H NMR (400 MHz, CD₂Cl₂) 6 8.25 (dd, J=9.7, 8.0 Hz, 2H), 7.84 (s, 2H, H₂O), 7.55-6.66 (m, 26H), 6.34 (dd, J=5.7, 3.3 Hz, 2H), 6.14 (dd, J=11.5, 7.4 Hz, 2H), 5.28-5.21 (m, 1H), 2.65 (dt, J=19.1, 3.3 Hz, 1H), 1.37-1.27 (m, 1H). ³¹P{¹H}NMR (162 MHz, CD₂Cl₂) 6 38.18 (s), 23.40 (s). Elem. Anal. Calcd for C₄₄H₃₇O₃P₂Re: C, 61.31; H, 4.33. Found: C, 61.08; H, 4.32.

### Example 3: Alkyne Metathesis Reactions

General Metathesis Procedure A. Alkyne metathesis reactions catalyzed by Re(≡CCH₂Ph)(^{Ph}PO)₂(H₂O)A Schlenk tube charged with a stir bar and 5 Å molecular sieves (MS) was heated with a heat gun (at 450° C.) under vacuum for over 5 min (to activate the MS). After cooling to room temperature, the Schlenk tube was evacuated and refilled with nitrogen three times. To the Schlenk tube were added substrate(s), Re(≡CCH₂Ph)(^{Ph}PO)₂(H₂O) (14), and distilled toluene. The mixture was stirred at specified temperatures for specified times. After the reaction, the mixture was filtered through Celite and washed thoroughly with dichloromethane. Evaporation of the volatiles on a rotary evaporator gave a residue, which was purified by column chromatography to afford the desired product.

### General Metathesis Procedure B.

Alkyne metathesis reaction catalyzed by the 1:0.5 mixture of Re(≡CCH₂Ph)(^{Ph}PO)₂(PMePh₂) and [(p-cymene)RuCl₂]₂. A Schlenk tube charged with a stir bar and 5 Å molecular sieves (MS) was heated with a heat gun (at 450° C.) under vacuum for over 5 min (to activate the MS). After cooling to room temperature, the Schlenk tube was evacuated and refilled with nitrogen three times. To the Schlenk tube were added substrate(s), Re(≡CCH₂Ph)(^{Ph}PO)₂(PMePh₂) (11), [(p-cymene)RuCl₂]₂ and distilled toluene. The mixture was stirred at specified temperatures for specified times. After the reaction, the mixture was filtered through Celite and washed thoroughly with dichloromethane. Evaporation of the volatiles on a rotary evaporator gave a residue, which was purified by column chromatography to afford the desired product.

### Example 3A: Alkyne Homometathesis Reactions

### 9-Octadecyne-1,18-diol

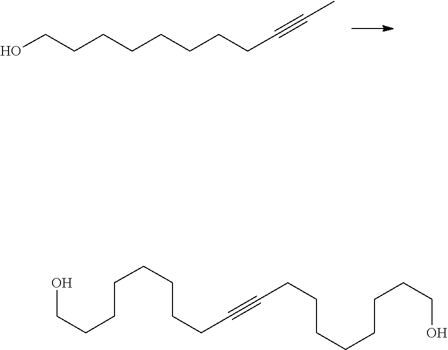 [SMILES: CC#CCCCCCCCCO ; OCCCCCCCCC#CCCCCCCCCO]

Synthesized following general procedure A. Undec-9-yn-1-ol (50.5 mg, 57 μL, 0.30 mmol), Re(≡CCH₂Ph)(^{Ph}PO)₂(H₂O) (12.9 mg, 0.015 mmol, 5 mol %), 5 Å molecular sieves (450 mg) and toluene (3 mL). 60° C., 2 h. Purified by column chromatography (Hexane:EA=1:1). White solid. Yield: 38.4 mg, 89%.

Synthesized following general procedure B. Undec-9-yn-1-ol (50.5 mg, 57 μL, 0.30 mmol), Re(≡CCH₂Ph)(^{Ph}PO)₂(PMePh₂) (15.7 mg, 0.015 mmol, 5 mol %), [(p-cymene)RuCl₂]2 (4.6 mg, 0.0075 mmol, 2.5 mol %), 5 Å molecular sieves (450 mg) and toluene (3 mL). 80° C., 4 h. The yield was determined by ¹H NMR using CH₂Ph₂ as the internal standard (75.6 mg, 75 μL, 0.45 mmol): 97%. The NMR data are the same as the reported ones.

### 4,4′-(Ethyne-1,2-diyl)dibenzaldehyde

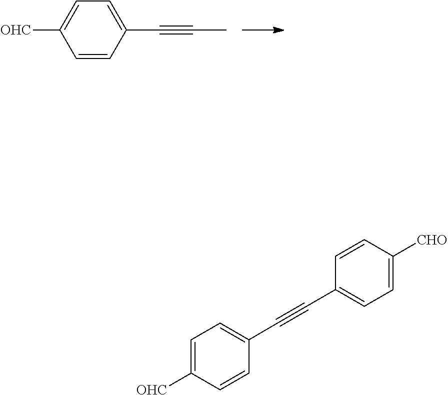 [SMILES: CC#Cc1ccc(C=O)cc1 ; O=Cc1ccc(C#Cc2ccc(C=O)cc2)cc1]

Synthesized following general procedure A. 4-(Prop-1-yn-1-yl)benzaldehyde (43.2 mg, 0.30 mmol), Re(≡CCH₂Ph)(^{Ph}PO)₂(H₂O) (12.9 mg, 0.015 mmol, 5 mol %), 5 Å molecular sieves (450 mg) and toluene (3 mL). 60° C., 4 h. The yield was determined by ¹H NMR using CH₂Ph₂ as the internal standard (75.6 mg, 75 μL, 0.45 mmol): 80%.

Synthesized following general procedure B. 4-(Prop-1-yn-1-yl)benzaldehyde (43.2 mg, 0.30 mmol), Re(≡CCH₂Ph)(^{Ph}PO)₂(PMePh₂) (15.7 mg, 0.015 mmol, 5 mol %), [(p-cymene)RuCl₂]₂ (4.6 mg, 0.0075 mmol, 2.5 mol %), 5 Å molecular sieves (450 mg) and toluene (3 mL). 80° C., 4 h. Purified by column chromatography (hexane:DCM=10:1). Pale yellow solid. Yield: 27.7 mg, 79%. The NMR data are the same as the reported ones.

### Example 3B: Alkyne Cross-Metathesis Reactions

### Tetradec-9-yn-1-ol

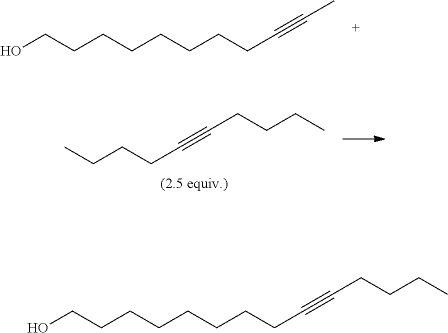

Synthesized following general procedure A. Undec-9-yn-1-ol (84.2 mg, 0.5 mmol), 5-octyne (172.9 mg, 1.25 mmol), Re(≡CCH₂Ph)(^{Ph}PO)₂(H₂O) (21.6 mg, 5 mol %), 5 Å molecular sieves (750 mg) and distilled toluene (5 mL). 60° C., 3 h. Purified by column chromatography (Hexane:EA=5:1). Pale-yellow oil. Yield: 78.0 mg, 74%.

Synthesized following general procedure B. Undec-9-yn-1-ol (84.2 mg, 0.5 mmol), 5-octyne (172.9 mg, 1.25 mmol), Re(≡CCH₂Ph)(^{Ph}PO)₂(PMePh₂) (26.1 mg, 0.025 mmol, 5 mol %), [(p-cymene)RuCl₂]2 (7.7 mg, 0.0125 mmol, 2.5 mol %), 5 Å molecular sieves (750 mg) and toluene (5 mL). 80° C., 5 h. Purified by column chromatography to afford the desired product as a pale-yellow oil. Yield: 78.9 mg, 75%. 1H NMR (400 MHz, CDCl₃) δ 3.58 (t, J=6.7 Hz, 2H), 2.19-2.00 (m, 4H), 1.89 (br, 1H), 1.59-1.48 (m, 2H), 1.48-1.14 (m, 14H), 0.87 (t, J=7.2 Hz, 3H). ¹³C{¹H}NMR (101 MHz, CDCl₃) δ 80.27, 80.21, 62.97, 32.81, 31.33, 29.40, 29.19 (2C), 28.84, 25.78, 21.99, 18.80, 18.50, 13.70. IR (ATR, cm⁻¹): 3331, 2929, 2857, 1464, 1434, 1056. HRMS (CI) Calcd. for [C₁₄H₂₆O]⁺ [M]⁺: 210.1984; Found: 210.1990.

### (4-((4-Methoxyphenyl])ethynyl)phenyl)(piperidin-1-yl)methanone

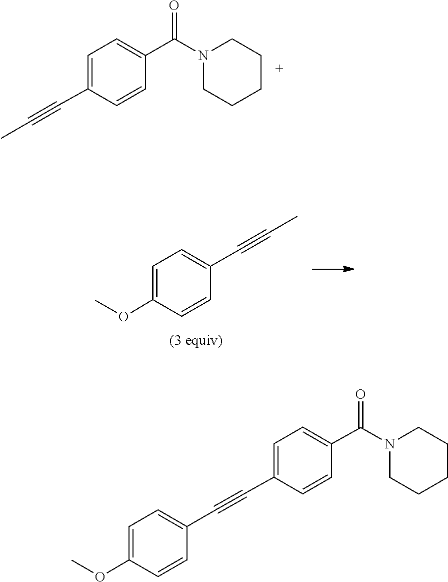 [SMILES: CC#Cc1ccc(C(=O)N2CCCCC2)cc1 ; CC#Cc1ccc(OC)cc1 ; COc1ccc(C#Cc2ccc(C(=O)N3CCCCC3)cc2)cc1]

Synthesized following general procedure A. Piperidin-1-yl(4-(prop-1-yn-1-yl)phenyl)methanone (68.2 mg, 0.3 mmol), 1-methoxy-4-(prop-1-yn-1-yl)benzene (131.6 mg, 0.9 mmol), Re(≡CCH₂Ph)(^{Ph}PO)₂(H₂O) (12.9 mg, 0.015 mmol, 5 mol %), 5 Å molecular sieves (1.80 g) and toluene (5 mL). 60° C., 4.5 h. Purified by column chromatography (hexane:EA=10:1 to 3:1). Pale brown crystals. Yield: 66.0 mg, 69%.

Synthesized following general procedure B. Piperidin-1-yl(4-(prop-1-yn-1-yl)phenyl)methanone (68.2 mg, 0.3 mmol), 1-methoxy-4-(prop-1-yn-1-yl)benzene (131.6 mg, 0.9 mmol), Re(≡CCH₂Ph)(^{Ph}PO)₂(PMePh₂) (15.7 mg, 0.015 mmol, 5 mol %), [(p-cymene)RuCl₂]₂ (4.6 mg, 0.0075 mmol), 5 Å molecular sieves (1.80 g) and toluene (6 mL). 80° C., 6 h. Purified by column chromatography (hexane:EA=10:1 to 3:1) to afford the desired product. Yield: 63.2 mg, 66%. ¹H NMR (400 MHz, CDCl₃) δ 7.52 (d, J=8.1 Hz, 2H), 7.46 (d, J=8.7 Hz, 2H), 7.36 (d, J=8.1 Hz, 2H), 6.87 (d, J=8.7 Hz, 2H), 3.82 (s, 3H), 3.69 (br, 2H), 3.33 (br, 2H), 1.67 (br, 4H), 1.51 (br, 2H). ¹³C{¹H}NMR (101 MHz, CDCl₃) δ 169.83, 159.89, 135.76, 133.23, 131.48, 127.04, 124.92, 115.06, 114.13, 90.82, 87.57, 55.41, 48.83 (br), 43.35 (br), 26.64 (br), 25.68 (br), 24.65. IR (ATR, cm⁻¹): 2999, 2934, 2854, 2214, 1624, 1600, 1568, 1515, 1434, 1279, 1247, 1174, 1137, 1106, 1026, 1000, 832. HRMS (ESI) Calcd. for [C₂₁H₂₁NNaO₂]⁺ [M+Na]⁺: 342.1465; Found: 342.1468.

### 4-(10-Bromodec-1-yn-1-yl)benzaldehyde

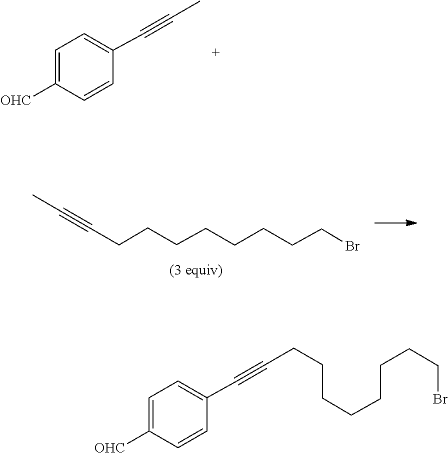 [SMILES: CC#Cc1ccc(C=O)cc1 ; CC#CCCCCCCCCBr ; O=Cc1ccc(C#CCCCCCCCCBr)cc1]

Synthesized following general procedure B. 4-(Prop-1-yn-1-yl)benzaldehyde (43.3 mg, 0.3 mmol), 11-bromoundec-2-yne (208.1 mg, 0.9 mmol), Re(≡CCH₂Ph)(^{Ph}PO)₂(PMePh₂) (15.7 mg, 0.015 mmol), [(p-cymene)RuCl₂]₂ (4.6 mg, 0.0075 mmol), 5 Å molecular sieves (1.80 g) and toluene (6 mL). 80° C., 5 h. Purified by column chromatography (hexane:EA=100:1). Colorless oil. Yield: 59.2 mg, 61%. 1H NMR (400 MHz, CDCl₃) δ 9.98 (s, 1H), 7.79 (d, J=7.9 Hz, 2H), 7.52 (d, J=8.0 Hz, 2H), 3.40 (t, J=6.8 Hz, 2H), 2.44 (t, J=7.0 Hz, 2H), 1.95-1.77 (m, 2H), 1.68-1.56 (m, 2H), 1.54-1.28 (m, 8H). ¹³C{¹H}NMR (101 MHz, CDCl₃) δ 191.64, 135.07, 132.21, 130.69, 129.63, 95.32, 80.31, 34.12, 32.89, 29.05, 28.91, 28.75, 28.56, 28.23, 19.67. IR (ATR, cm⁻¹): 2928, 2854, 2729, 2226, 1699, 1600, 1561, 1206, 1164, 828. HRMS (CI) Calcd. for [C₁₇H₂₂BrO]⁺ [M+H]⁺: 321.0849; Found: 321.0858.

### Example 3C—Ring-closing Alkyne Metathesis

### 1,8-Dioxacyclotetradec-11-yne-2,7-dione

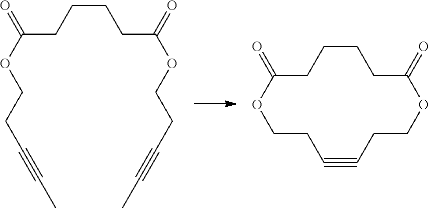 [SMILES: CC#CCCOC(=O)CCCCC(=O)OCCC#CC ; O=C1CCCCC(=O)OCCC#CCCO1]

Synthesized following general procedure A. Di(pent-3-yn-1-yl) adipate (83.5 mg, 0.30 mmol), Re(≡CCH₂Ph)(^{Ph}PO)₂(H₂O) (12.9 mg, 0.015 mmol), 5 Å molecular sieves (1.0 g) and toluene (60 mL). 80° C., 8 h. Purified by column chromatography (hexane:EA=5:1). White needle crystals. Yield: 62.0 mg, 92%.

Synthesized following general procedure B. Di(pent-3-yn-1-yl) adipate (83.5 mg, 0.30 mmol), Re(≡CCH₂Ph)(^{Ph}PO)₂(PMePh₂) (15.7 mg, 0.015 mmol), [(p-cymene)RuCl₂]₂(4.6 mg, 0.0075 mmol), 5 Å molecular sieves (1.0 g) and toluene (60 mL). 80° C., 8 h. Purified by column chromatography (hexane:EA=5:1) to afford the desired product. Yield: 62.8 mg, 93%. 1H NMR (400 MHz, CDCl₃) δ 4.13 (t, J=5.4 Hz, 4H), 2.57-2.46 (m, 4H), 2.43-2.32 (m, 4H), 1.77-1.72 (m, 4H). ¹³C{¹H}NMR (101 MHz, CDCl₃) δ 173.28, 78.03, 62.67, 35.08, 25.12, 19.24. HRMS (ESI) Calcd. for [C₁₂H₁₆NaO₄]⁺ [M+Na]⁺: 247.0941; Found: 247.0947.

### Azacyclononadec-10-yn-2-one

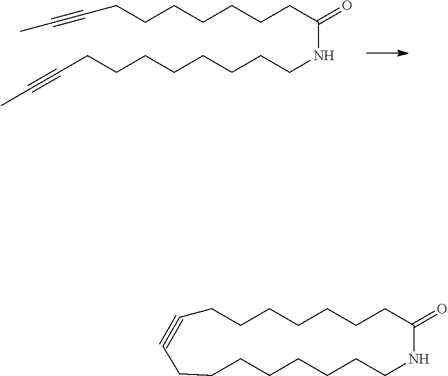 [SMILES: CC#CCCCCCCCCNC(=O)CCCCCCCC#CC ; O=C1CCCCCCCC#CCCCCCCCCN1]

Synthesized following general procedure B. N-(undec-9-yn-1-yl)undec-9-ynamide (66.3 mg, 0.20 mmol), Re(≡CCH₂Ph)(^{Ph}PO)₂(PMePh₂) (20.9 mg, 0.02 mmol), [(p-cymene)RuCl₂]₂ (6.1 mg, 0.01 mmol), 5 Å molecular sieves (0.60 g) and toluene (40 mL). 100° C., 12 h. Purified by column chromatography (hexane:EA=2:1). White crystals. Yield: 38.0 mg, 68%. 1H NMR (400 MHz, CDCl₃) δ 5.56 (br, 1H), 3.30 (td, J=5.8, 5.7 Hz, 2H), 2.25-2.06 (m, 6H), 1.71-1.57 (m, 2H), 1.55-1.46 (m, 2H), 1.46-1.36 (m, 8H), 1.38-1.27 (m, 1OH).

### Example 3D: Acyclic Diyne Metathesis Macrocyclization (ADIMAC)

### Macrocycle 32

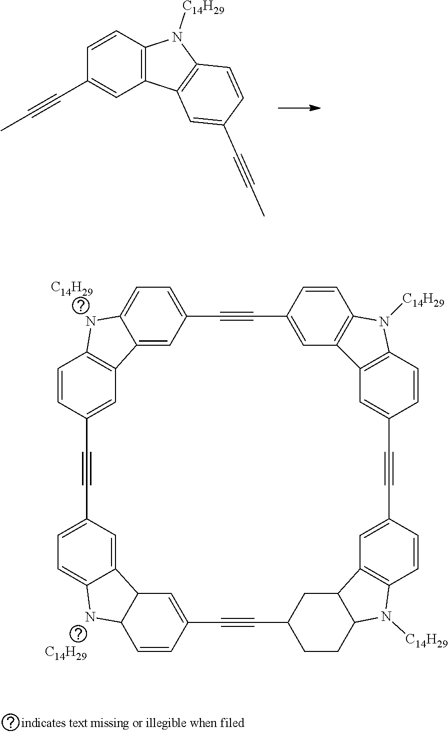

Synthesized following general procedure A. 3,6-Di(prop-1-yn-1-yl)−9-tetradecylcarbazole (83.5 mg, 0.30 mmol), Re(≡CCH₂Ph)(^{Ph}PO)₂(H₂O) (8.6 mg, 0.01 mmol), 5 Å molecular sieves (0.60 g) and toluene (10 mL). 60° C., 14 h. Purified by column chromatography (hexane:chloroform=1:1). White solid. Yield: 38.2 mg, 99%.

Synthesized following general procedure B. 3,6-Di(prop-1-yn-1-yl)−9-tetradecylcarbazole (44.0 mg, 0.10 mmol), Re(≡CCH₂Ph)(^{Ph}PO)₂(PMePh₂) (10.4 mg, 0.01 mmol), [(p-cymene)RuCl₂]₂ (3.1 mg, 0.005 mmol), 5 Å molecular sieves (0.60 g) and toluene (10 mL). 80° C., 20 h. Purified by column chromatography (hexane:chloroform=1:1) to afford the desired product. Yield: 38.0 mg, 98%. 1H NMR (400 MHz, CDCl₃) δ 8.37 (d, J=1.0 Hz, 2H), 7.71 (dd, J=8.4, 1.4 Hz, 2H), 7.36 (d, J=8.5 Hz, 2H), 4.27 (t, J=7.0 Hz, 2H), 1.95-1.80 (m, 2H), 1.46-1.13 (m, 22H), 0.88 (t, J=6.8 Hz, 3H). ¹³C{¹H}NMR (101 MHz, CDCl₃) δ 140.24, 129.38, 124.06, 122.77, 114.55, 109.00, 89.23, 43.45, 32.08, 29.85, 29.81, 29.78, 29.73, 29.68, 29.53, 29.15, 27.46, 22.85, 14.29.

### Example 3E—Ring-opening Alkyne Metathesis Polymerization

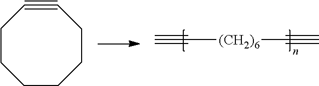

A mixture of Re(≡CCH₂Ph)(^{Ph}PO)₂(H₂O) (8.6 mg, 0.01 mmol), cyclooctyne (108 mg, 1 mmol) and 1 mL of toluene was heated at 60° C. for 1 hour. After the reaction, to the mixture was added 10 ml of methanol to precipitate a white solid, which was collected by filtration and dried under vacuum to afford the desired product. Yield: 94 mg. 87%. ¹H NMR (400 MHz, CDCl₃) δ 2.13 (br, 4H), 1.47 (br, 4H), 1.37 (br, 4H). ¹³C{¹H}NMR (101 MHz, CDCl₃) δ 80.29, 29.19, 28.53, 18.86.

What is claimed is:

1. A rhenium(V) alkylidyne complex of Formula 1: 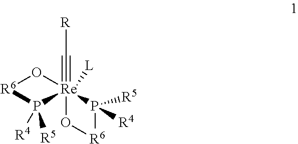
wherein L is H₂O, an ether, a ketone, a furan, a thioether, a thiophene, a tertiary amine, or a metal salt selected from the group consisting of a metal halide, a metal alkoxide, a metal thiolate and a metal amide
R is alkyl, aryl, thiophene, or CH₂Ari, wherein Ar¹ is aryl;
each of R⁴ and R⁵ is independently alkyl, cycloalkyl or aryl; and
R⁶ for each instance is independently alkyl, alkenyl or aryl.

2. The rhenium(V) alkylidyne complex of claim 1, wherein Lis H₂O, tetrahydrofuran, tetrahydropyran, or Et₂O.

3. The rhenium(V) alkylidyne complex of claim 1, wherein R is CH₂Ari, wherein Ar¹ is phenyl or o-bromophenyl.

4. The rhenium(V) alkylidyne complex of claim 1, wherein each of R⁴ and R⁵ is independently alkyl, cycloalkyl, or phenyl optionally substituted with one or more substituents selected from alkyl, CH₃, OCH₃, F, and CF₃.

5. The rhenium(V) alkylidyne complex of claim 1, wherein each of R⁴ and R⁵ is cyclohexyl, phenyl, 4-fluorophenyl, 2,4-difluorophenyl, 4-trifluoromethylphenyl, 2,4-bis(trifluoromethyl)phenyl, 2,4-dimethylphenyl, or 2,4-dimethoxyphenyl.

6. The rhenium(V) alkylidyne complex of claim 1, wherein R⁶ is phenyl optionally substituted with alkyl or trifluoromethyl.

7. The rhenium(V) alkylidyne complex of claim 1, wherein R⁶ is a moiety selected from the group consisting of: 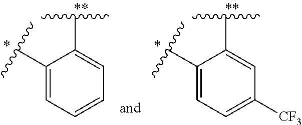 [SMILES: *c1ccccc1* ; *c1ccc(C(F)(F)F)cc1*]
wherein * represents the site of the covalent bond to oxygen and ** represents the site of the covalent bond to phosphorous.

8. The rhenium(V) alkylidyne complex of claim 1, wherein the rhenium(V) alkylidyne complex is selected from the group consisting of: 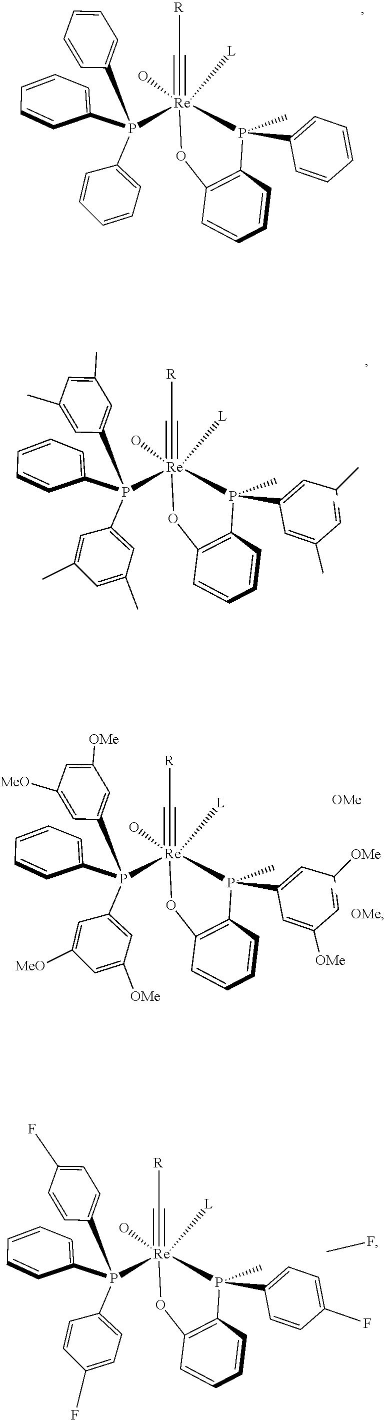 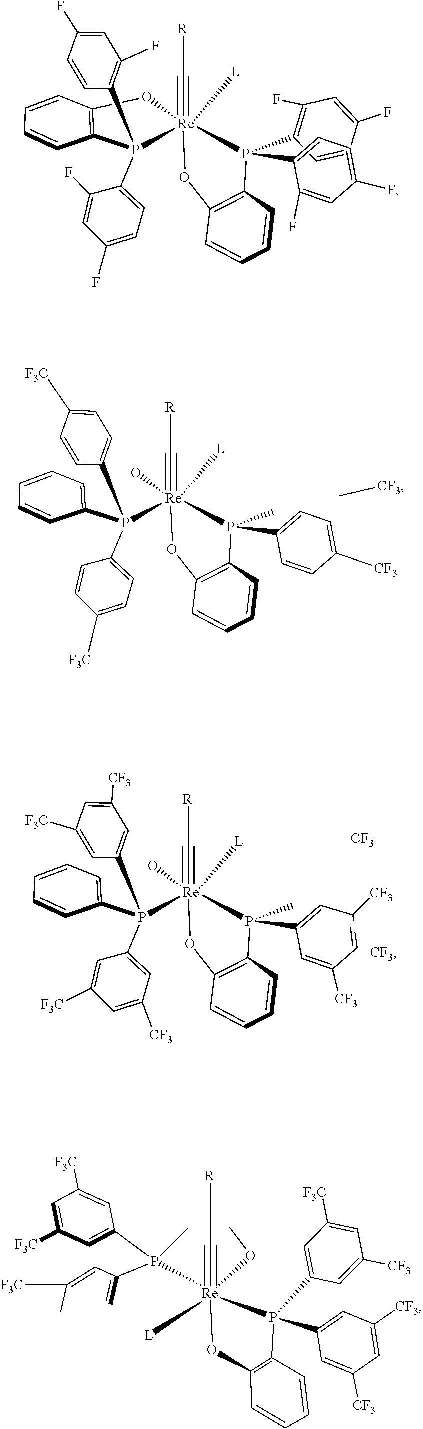 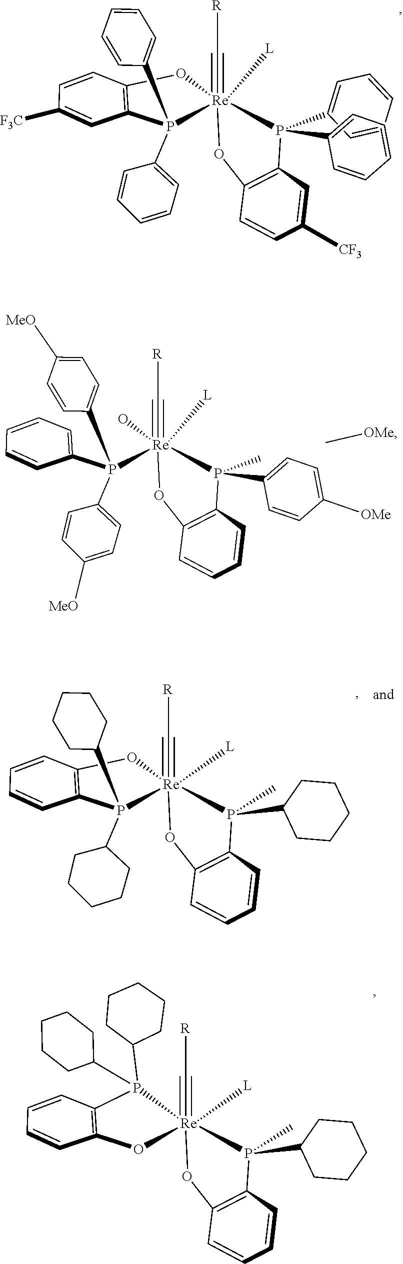
wherein R is alkyl, aryl, 2-thiophene, or CH₂Ar^{O}, wherein Ar¹ is phenyl or o-bromophenyl; and L is H₂O, tetrahydrofuran, tetrahydropyran, or Et₂O.

9. The rhenium(V) alkylidyne complex of claim 8, wherein R is CH₂Ph.

10. The rhenium(V) alkylidyne complex of claim 9, wherein L is H₂O.

11. The rhenium(V) alkylidyne complex of claim 1, wherein the rhenium(V) alkylidyne complex has the structure:

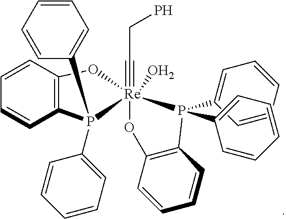

12. A method of preparing the rhenium (V) alkylidyne complex of claim 1, the method comprising: contacting a catalyst precursor of Formula 2: 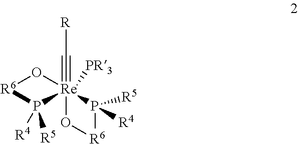
wherein R is alkyl, aryl, thiophene, or CH₂Ar¹, wherein Ar¹ is aryl;
PR′₃ is PMePh₂ or PMe₂Ph;
each of R⁴ and R⁵ is independently alkyl, cycloalkyl, or aryl; and
R⁶ for each instance is independently alkyl, alkenyl or aryl;
with a ligand selected from the group consisting of H₂O and an alkyl ether; and a phosphine scavenger selected from CuCl, CuI, or [(p-cymene]RuCl₂]₂; thereby forming the rhenium (V) alkylidyne complex.

13. A method for performing a metathesis reaction, the method comprising: contacting a rhenium(V) alkylidyne complex of claim 1 with at least one alkyne substrate thereby forming an alkyne metathesis product.

14. The method of claim 13, wherein the at least one alkyne substrate is selected from the group consisting of an acyclic alkyne, a cyclic alkyne, an acyclic diyne, and a cyclic diyne.

15. A method for performing a metathesis reaction, the method comprising: contacting a catalyst precursor of Formula 2, a phosphine scavenger, and at least one alkyne substrate thereby forming an alkyne metathesis product, wherein the catalyst precursor of Formula 2 has the structure: 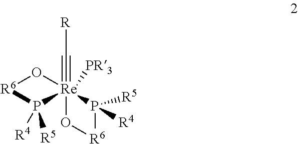
wherein R is alkyl, aryl, thiophene, or CH₂Ar¹, wherein Ar¹ is aryl;
PR′₃ is PMePh₂ or PMe₂Ph;
each of R⁴ and R⁵ is independently alkyl, cycloalkyl, or aryl; and
R⁶ for each instance is independently alkyl, alkenyl or aryl; and
the phosphine scavenger is selected from [(p-cymene]RuCl₂]₂, [(p-cymene]OsCl₂]₂, [RhCl(COD)]₂, IrCl(COD)]₂, PdCl₂(COD), CuCl, or CuI.

16. The method of claim 15, wherein R is CH₂ArO, wherein Ar¹ is phenyl or o-bromophenyl.

17. The method of claim 15, wherein each of R⁴ and R⁵ is cyclohexyl, phenyl, 4-fluorophenyl, 2,4-difluorophenyl, 4-trifluoromethylphenyl, 2,4-bis(trifluoromethyl)phenyl, 2,4-dimethylphenyl, or 2,4-dimethoxyphenyl.

18. The method of claim 15, wherein R⁶ is phenyl optionally substituted with alkyl or trifluoromethyl.

19. The method of claim 15, wherein R⁶ is a moiety selected from the group consisting of: 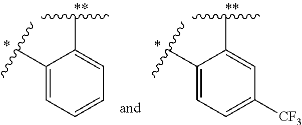 [SMILES: *c1ccccc1* ; *c1ccc(C(F)(F)F)cc1*]
wherein * represents the site of the covalent bond to oxygen and ** represents the site of the covalent bond to phosphorous.

20. The method of claim 15, wherein the phosphine scavenger is [(p-cymene]RuCl₂]2 and the catalyst precursor is: [chemical structure]
